SEQUENCE LISTING (3 sequences)

SEQ ID NO: 1 - protein, 13 aa, unknown
  polyhistidine tag
MGHHHHHHSGSEF

SEQ ID NO: 2 - protein, 134 aa, homo sapiens
TGMPVSAFTVILSKAYPAIGTPIPFDKILYNRQQHYDPRTGIFTCQIPGIYYFSYHVHVKGTHVWVGLYKNGTPVMYTYDEYTKGYLDQASGSAIIDLTENDQVWLQLPNAESNGLYSSEYVHSSFSGFLVAPM

SEQ ID NO: 3 - protein, 134 aa, Mouse
TGMPVSAFTVILSKAYPAVGAPIPFDEILYNRQQHYDPRSGIFTCKIPGIYYFSYHVHVKGTHVWVGLYKNGTPTMYTYDEYSKGYLDQASGSAIMELTENDQVWLQLPNAESNGLYSSEYVHSSFSGFLVAPM

(19) United States
(12) Patent Application Publication
(10) Pub. No.: US 2021/0223263 A1
(43) Pub. Date: Jul. 22, 2021

(54) Type X Collagen Assays and Methods of Use Thereof

(71) Applicant: Shriners Hospitals for Children, Tampa, FL (US)

(72) Inventors: William A. Horton, Portland, OR (US); Gregory P. Lunstrum, Portland, OR (US); Ryan F. Coghlan, Portland, OR (US); Jon A. Oberdorf, Warren, OR (US)

(21) Appl. No.: 17/215,629

(22) Filed: Mar. 29, 2021

Related U.S. Application Data
(63) Continuation of application No. 16/990,052, filed on Aug. 11, 2020, now abandoned.
Application No. 16/990,052 is a continuation of application No. 15/915,566, filed on Mar. 8, 2018, now abandoned.
(60) Provisional application No. 62/469,053, filed on Mar. 9, 2017.
(60) Provisional application No. 62/588,789, filed on Nov. 20, 2017.

(51) Int. Cl. G01N 33/68; G01N 33/50; G01N 33/543; G01N 33/566; C07K 14/78; G01N 33/535; G01N 33/574

(52) U.S. Cl. CPC G01N 33/6887; G01N 33/5044; G01N 33/5082; G01N 33/5029; G01N 33/54306; G01N 33/566; A61K 38/00; G01N 33/535; G01N 33/57488; G01N 2800/52; G01N 2800/10; G01N 2800/60; C07K 14/78

(57) ABSTRACT

The present invention provides methods for determining bone growth velocity comprising: (a) measuring an amount of a collagen X marker in a sample obtained from a subject in need thereof; and (b) comparing the amount of collagen X marker measured in step (a) with a collagen X marker standard curve, wherein the amount of collagen X marker is measured using at least two reagents. In an embodiment, there is at least one capture reagent and at least one detection reagent. In a preferred embodiment for measuring CXM, the capture reagent is the aptamer SOMA1 and the detection reagent is the monoclonal antibody mAb X34. The present invention further provides methods for treating diseases, disorders or conditions comprising receiving an identification of an amount of CXM in a sample, wherein the amount of CXM has been identified using a combination of SOMA1 and mAb X34 as CXM-binding reagents, and administering a treatment in light of the amount of CXM in the sample.

This application claims priority benefit of U.S. Provisional Application Nos. 62/469,053 filed Mar. 9, 2017 and 62/588,789 filed Nov. 20, 2017, each of which is incorporated by reference herein in its entirety.

This invention was made with government support under grant no. R21AR065657 awarded by the National Institutes of Health. The government has certain rights in the invention.

### FIELD OF THE INVENTION

The instant disclosure relates to methods for measuring bone growth velocity by measuring a collagen X marker. The collagen X marker is a stable trimeric degradation fragment of type X collagen and functions as a real-time marker for bone growth velocity.

### BACKGROUND OF THE INVENTION

Growth is an integral component of human development. Clinically, it typically refers to skeletal growth measured in infants as body length and as height in children and adolescents. It reflects the dynamic process of endochondral ossification that occurs in growth plates that reside in all bones that contribute to increasing length and height.

Growth is often used as a nonspecific indicator of health in childhood. Indeed, most serious illnesses in children are associated with reduced growth, which may be restored to normal with successful treatment. Many childhood diseases, typically endocrine disorders, specifically impact growth by affecting hormones and growth factors that regulate bone growth. Another large group of childhood growth disorders, the skeletal dysplasias, reflect genetic disturbances in the bone growth machinery. Publications reflecting the state of the art of skeletal bone growth include: F. Long et al., “Development of the endochondral skeleton,” Cold Spring Harb Perspect Biol 5, a008334 (2013); K. Yeung Tsang et al., “The chondrocytic journey in endochondral bone growth and skeletal dysplasia,” Birth Defects Res C Embryo Today 102, 52-73 (2014); W. A. Horton et al., “International workshop on the Skeletal Growth Plate,” Stevenson, Wash., Jun. 11-15, 2006, Matrix Biol 26, 324-329 (2007); H. M. Kronenberg, “Developmental regulation of the growth plate,” Nature 423, 332-336 (2003); M. de Onis et al., “Childhood stunting: a global perspective,” Matern Child Nutr 12 (Suppl 1), 12-26 (2016); J. Baron et al., “Short and tall stature: a new paradigm emerges,” Nat Rev Endocrinol 11, 735-746 (2015); S. Melmed et al., Williams Textbook of Endocrinology (Elsevier, Philadelphia, ed. 13, 2016); L. Bonafe et al., “Nosology and classification of genetic skeletal disorders: 2015 revision,” Am J Med Genet A 167A, 2869-2892 (2015), each of which is incorporated by reference herein.

Measuring static parameters of growth, such as body length or height, is relatively simple. In contrast, measuring growth rate or velocity, the key parameter for evaluating and managing growth disturbances, is much more challenging because skeletal growth is a slow process and measurement techniques lack the precision to accurately detect these small changes. The accepted practice measures length, height and other anthropometric parameters at 6 or 12 month intervals typically using a calibrated measuring device, such as a stadiometer, and calculates annualized velocity accordingly (cm/year). Further complicating this approach, especially in infants, are difficulties positioning patients to achieve maximal lengths and completely excluding observer subjectivity.

Despite concerns over the reliability of short term stadiometer-based height velocity determination, this practice has become established for monitoring the growth of healthy children. Such practices have been discussed in, for example, J. M. Tanner et al., “Clinical longitudinal standards for height, weight, height velocity, weight velocity, and stages of puberty,” Arch Dis Child 51, 170-179 (1976); L. D. Voss et al., “The reliability of height measurement (the Wessex Growth Study),” Arch Dis Child 65, 1340-1344 (1990); L. D. Voss et al., “The reliability of height and height velocity in the assessment of growth (the Wessex Growth Study),” Arch Dis Child 66, 833-837 (1991); J. Van den Broeck et al., “Validity of height velocity as a diagnostic criterion for idiopathic growth hormone deficiency and Turner syndrome,” Horm Res 51, 68-73 (1999); T. M. Schmid et al., “A short chain (pro)collagen from aged endochondral chondrocytes, biochemical characterization,” J Biol Chem 258, 9504-9509 (1983); T. M. Schmid et al., “Immunohistochemical localization of short chain cartilage collagen (type X) in avian tissues,” J Cell Biol 100, 598-605 (1985); T. F. Linsenmayer et al., “Type X collagen: a hypertrophic cartilage-specific molecule,” Pathol Immunopathol Res 7, 14-19 (1988), each of which is incorporated by reference herein. Stadiometer-based velocity determination is much less acceptable for managing pediatric growth disturbances, especially for assessing responses to interventions designed to improve growth and health. Thus, there is a clear need for a means to accurately measure bone growth velocity on a time frame much shorter than is currently available.

### SUMMARY OF THE INVENTION

This invention provides a method for determining bone growth velocity by (a) measuring an amount of CXM in a subject in need thereof, and comparing the amount of CXM measured in step (a) with a CXM standard curve. In an embodiment, the amount of CXM is measured by using a combination of an aptamer and an antibody, such as SOMA1 and mAb X34, as CXM-binding reagents. In an embodiment, these reagents, such as SOMA1 and mAb X34, may be used in a solid phase binding assay or a multiplex assay. In a preferred embodiment, SOMA1 is used as a capture reagent and mAb X34 is used as a detection reagent. Measuring the amount of CXM provides a real-time reading of bone growth plate activity that is correlated with skeletal bone growth velocity at the time when the sample was taken from the subject.

This invention provides a method for quantification of the amount of CXM in a sample obtained from a subject, comprising: (a) contacting the sample obtained from the subject with biotinylated SOMA1 immobilized on a streptavidin-coated plate; (b) removing material in the sample not bound by SOMA1 in step (a); and (c) detecting immobilized CXM using mAb X34 conjugated with horseradish peroxidase (HRP), or detected with an HRP-labeled secondary antibody, wherein an HRP signal reflects the amount of CXM in the sample obtained from the subject.

This invention provides a method for determining bone growth velocity comprising: (a) measuring an amount of Cxm in a sample obtained from a subject in need thereof; and (b) comparing the amount of Cxm measured in step (a) with a Cxm standard curve, wherein the amount of Cxm is measured using a combination of an aptamer and an antibody as Cxm-binding reagents. In an embodiment, preferred reagents, such as SOMA1 and mAb X34, may be used in a solid phase binding assay or in a multiplexed assay. In an embodiment, SOMA1 is used as a capture reagent and mAb X34 is used as a detection reagent. Determining the amount of Cxm provides a real-time reading of bone growth plate activity that is correlated with skeletal bone growth velocity at the time when the sample was taken from the subject.

This invention provides a method for quantification of Cxm in a sample obtained from a subject comprising: (a) contacting the sample with immobilized SOMA1 so as to capture Cxm bound to SOMA1 in a Cxm-SOMA1 complex; (b) contacting the Cxm-SOMA1 complex formed in step (a) with an antibody conjugated with a reporter molecule; and (c) detecting a reporter signal from the reporter molecule, wherein the reporter signal reflects the amount of Cxm in the sample from the subject.

This invention provides methods for measuring CXM in order to monitor or detect: a bone growth response in disorders of bone growth and other conditions in which bone growth is disturbed; idiopathic scoliosis; bone fracture healing; osteoarthritis; cancer; or heterotopic ossification, wherein the measurements occur before, during and/or after an intervention or treatment. This invention provides methods for determining whether and/or when bone growth has stopped at the end of puberty, so as to provide guidance on whether and/or when to stop treating a bone growth disorder with growth promoting agents.

This invention provides methods for treating diseases, disorders or conditions in a human subject comprising: (a) receiving an identification of the human subject as having an amount of CXM in a sample obtained from the human subject, wherein the amount of CXM has been identified by a method comprising using a combination of SOMA1 and mAb X34 as CXM-binding reagents; and (b) administering a treatment to the human subject identified as having the amount of CXM in the sample.

In an embodiment, the sample is a blood sample, a serum sample, a plasma sample, or a dried blood spot. In an embodiment, SOMA1 and mAb X34 are used to bind CXM in a solid phase binding assay. In an embodiment, the solid phase binding assay uses SOMA1 as a capture reagent and mAb X34 as a detection reagent. In an embodiment, the capture reagent is immobilized on a solid phase support. In an embodiment, the detection reagent is linked to a reporter molecule, further wherein the reporter molecule is selected from the group consisting of horseradish peroxidase (HRP), alkaline phosphatase, luciferase, a chemical fluorophore, a quantum dot fluorescent reporter molecule, a Raman reporter molecule, a Maverick Detection System reporter molecule, an electrochemical immunosensor reporter molecule, an aptosensor reporter molecule, a mass spectrometry reporter molecule, an sAB-colloidal gold conjugate reporter molecule, and a DNA-directed immobilization reporter molecule.

In an embodiment, the amount of CXM identified in the sample provides a real-time readout of bone growth plate activity that is correlated with skeletal bone growth velocity at the time of sampling. In an embodiment, the disease, disorder or condition is selected from the group consisting of rickets, hypogonadism, growth hormone deficiency, intrauterine growth retardation, Russell Silver Syndrome, vitamin D deficiency, idiopathic skeletal hyperostosis, osteoporosis, and cancer. In an embodiment, the treatment is selected from the group consisting of growth hormone therapy, C-type natriuretic peptide (CNP) therapy, bone morphogenetic protein (BMP) therapy, insulin-like growth factor 1 (IGF-1) therapy, FGFR3 antagonist therapy, and vosoritide (BMN 111) therapy.

### BRIEF DESCRIPTION OF THE DRAWINGS

FIGS. 1A and 1B depict mammalian type X collagen.

FIGS. 2A and 2B depict the identification and subunit characterization of the CXM marker.

FIGS. 3A and 3B depict a mass spectrometry analysis of the CXM marker.

FIGS. 4A, 4B and 4C depict western blots showing that the CXM marker decreases with age and can be detected in human urine and mouse blood.

FIGS. 5A, 5B and 5C depict the correlation of tail and long bone growth velocities with Cxm serum concentrations in mice.

FIGS. 6A, 6B, 6C and 6D depict the correlation of CXM with age and growth velocity.

FIG. 7 shows that CXM concentration increases during adult fracture healing.

FIG. 8 plots the diurnal variation of CXM.

FIG. 9 depicts a mass spectrometry analysis of fully tryptic CXM fragments.

FIG. 10 depicts the dissociation of trimeric mouse rNC1 into dimers and monomers.

FIG. 11 depicts lower limit of quantitation (LLOQ) testing of CXM.

FIGS. 12A, 12B and 12C plot CXM stability testing, showing variances from repeated freeze-thaw cycles, temperature stresses and storage conditions.

FIGS. 13A, 13B and 13C depict the relationship of stadiometer-based height velocities to CXM.

FIGS. 14A, 14B and 14C depict the relationships among serum, plasma and DBS CXM concentrations.

FIG. 15 plots that half-life of Cxm.

FIG. 16 is a table depicting the technical characterization of CXM assay.

FIG. 17 is a table depicting diurnal variation data.

FIG. 18 is a table depicting blood sample data.

### DETAILED DESCRIPTION OF THE INVENTION

The following description is presented to enable a person of ordinary skill in the art to make and use the various embodiments. Descriptions of specific methods, compositions, techniques, and applications are provided only as examples. Various modifications to the examples described herein will be readily apparent to those of ordinary skill in the art, and the general principles described herein may be applied to other examples and applications without departing from the spirit and scope of the various embodiments. Thus, the various embodiments are not intended to be limited to the examples described herein and shown, but are to be accorded the scope consistent with the claims.

Unless defined otherwise, all technical and scientific terms used herein have the same meaning as is commonly understood by one of ordinary skill in the art to which this invention belongs.

As used herein, “CXM” refers to the collagen X marker in humans. This bone growth velocity marker is the trimeric non-collagenous 1 (NC1) domain of type X collagen.

As used herein, “Cxm” refers to collagen X marker in other subjects, excluding humans. This bone growth velocity marker is the trimeric non-collagenous 1 (NC1) domain of type X collagen in non-human subjects.

As used herein, “bone growth velocity” refers to the change in bone growth of a given body unit per unit time. For example, it can refer to the extent that bones grow in length, either individually or in the aggregate, per unit time. The body unit measurement can be overall body length (e.g., in the case of an infant), or height, arm span, or upper or lower body segment. The unit time is typically one year. Most commonly, “bone growth velocity” refers to the change in length for infants and height for children per year.

As used herein, “mAb” refers to a monoclonal antibody.

As used herein, various “binding reagent(s)” may be used in the assays in accordance with the present invention. The binding reagents in accordance with the present invention may be capture reagents and/or detection reagents.

As used herein, a “reporter molecule” may be linked to a detection reagent. For example, a reporter molecule may be horseradish peroxidase (HRP), alkaline phosphatase, luciferase, a chemical fluorophore, a quantum dot fluorescent reporter molecule, a Raman reporter molecule, a Maverick Detection System reporter molecule (https://www.genalyte.com/about-us/our-technology), an electrochemical immunosensor reporter molecule, an aptosensor reporter molecule, a mass spectrometry reporter molecule, an sAB-colloidal gold conjugate reporter molecule, and/or a DNA-directed immobilization reporter molecule.

As used herein, “subject” refers to vertebrates. For example, a vertebrate may be a mammal such as, without limitation, a human, a mouse, a rat, a dog, a monkey, a horse, a goat, a sheep or a guinea pig.

As used herein, “sample” refers to a blood sample, serum sample, plasma sample or a dried blood spot obtained from a subject.

In an aspect, the present invention provides a method for determining bone growth velocity comprising: (a) measuring an amount of a collagen X marker in a sample obtained from a subject in need thereof; and (b) comparing the amount of collagen X marker measured in step (a) with a collagen X marker standard curve, wherein the amount of collagen X marker is measured using at least one reagent. In an embodiment, there is at least one capture reagent, for example, at least one aptamer reagent, and at least one detection reagent, for example, at least one antibody reagent. In an embodiment, the collagen X marker is CXM. In an embodiment, the collagen X marker is Cxm.

The detection of CXM in humans and the corresponding collagen X marker in other species (Cxm in non-human vertebrates) can be accomplished in a variety of ways, for example, as described in Nimse et al., “Biomarker detection technologies and future directions,” Analyst 141, 740-755 (2016), which is incorporated herein by reference. In accordance with an embodiment of the present invention, CXM can be detected analogously to PSA as described in Nimse. In an embodiment, the method is performed using an enzyme-linked immunosorbent assay (ELISA). The various components for performing ELISAs are generally well known in the art and can be purchased from commercially available sources. Some example components for an ELISA as used in accordance with the present invention may include, but are not limited to, 96 well EIA/RIA high-binding plate (Costar #3590), immuno-pure streptavidin (Thermo #21125), superblock blocking buffer (Thermo #37515), BSA for coating plates (RMBIO #BSA-BAF-01K), BSA for assay solutions (Gold Biotechnology #A-421-100), Tween-20 (Fisher #BP337-500), dextran sulfate sodium salt (Sigma #31404-25G-F). Calibrators for assays can be, for example, rNC1 proteins obtained from BioMatik. Suitable procedures for performing assays may be found in the Examples herein and in, for example, T. W. McDade, J. Burhop, J. Dohnal, “High-sensitivity enzyme immunoassay for C-reactive protein in dried blood spots,” Clin Chem 50, 652-654 (2004), which is incorporated by reference herein.

In another embodiment, a method of the invention is performed in a multiplex assay format, such in a planar microchip array or in a microsphere suspension. Multiplex assays are well known in the art. For example, see Ellington et al., “Antibody-based protein multiplex platforms: technical and operational challenges,” Clinical Chemistry 56, 186-193 (2010), which is incorporated by reference herein. Additionally, Luminex® assays have been described. See, for example, Dunbar, “Applications of Luminex® xMAP™ technology for rapid, high throughput multiplexed nucleic acid detection,” Clinica Chemica Acta 363, issues 1-2, pp. 71-82 (January 2006), which is incorporated by reference herein. Components for multiplex assays are also known in the art and can be purchased from commercially available sources. See, for example, “Multiplex assays for the Luminex instrument platform” (https://www.thermofisher.com/content/dam/LifeTech/global/promotions/global/images/aai-2015/aai-pdfs/CO123353-Luminex-brochure.PDF). The amount of a collagen X marker in a sample may be measured by contacting the sample obtained from the subject with capture reagent immobilized on a solid plate; (b) removing material in the sample not bound by capture reagent in step (a); and (c) detecting immobilized collagen X marker using detection reagent. In an embodiment, the collagen X marker is CXM. In an embodiment, the collagen X marker is Cxm.

In an embodiment, the capture reagent is an aptamer or an antibody immobilized on a solid phase support. An aptamer such as SOMA1 is preferred. In an embodiment, the solid phase support may be a 96 well plastic plate. In an embodiment, the capture reagent is an aptamer, such as SOMA1, which has been biotinylated and immobilized on a streptavidin-coated plate.

In an embodiment, the detection reagent is an aptamer or an antibody. In an embodiment, the detection reagent is a type X collagen antibody. In an embodiment, the type X collagen antibody is mouse anti-human monoclonal antibody X34 (mAb X34) as disclosed in I. Girkontaite et al., “Immunolocal-ization of type X collagen in normal fetal and adult osteoarthritic cartilage with monoclonal antibodies,” Matrix Biol 15, 231-238 (1996), which is incorporated by reference herein. In an embodiment, the Cxm detection reagent is an avian polyclonal antibody raised against mouse rNC1 obtained from Ayes Labs, Inc. In an embodiment, the detection reagent is a chicken anti-mouse-rNC1 antibody. In an embodiment, the detection reagent is a rabbit polyclonal antibody (pAb) against human rNC1 (USCNK #PAC156Hu01). In an embodiment, the detection reagent is a rabbit pAb raised against mouse rNC1 (USCNK #PAC156Mo01). In an embodiment, the detection reagent is an aptamer. For example, an aptamer such as SOMA1, or similar aptamers, can also be used for detection in addition to their use as capture reagents. In some embodiments, a type X collagen antibody, or an aptamer, may be conjugated to a reporter molecule. In an embodiment, the type X collagen antibody may be conjugated to a chemical fluorophore, including but not limited to, R-phycoerythrin. In an embodiment, the type X collagen antibody may be conjugated to horseradish peroxidase (HRP, Southern Biotech). In an embodiment, the type X collagen antibody is detected using an HRP-labeled secondary antibody such as goat anti-rabbit (Amersham #NA934V) or goat anti-chicken (Ayes Labs, Inc. #H-1004). In an embodiment, the type X collagen antibody may be covalently coupled to agarose using an AminoLink Plus immobilization kit (Thermo #44894).

In an embodiment, the capture reagent is an aptamer or an antibody. In an embodiment, the capture reagent is an aptamer (also known as a slow off-rate modified aptamer, or SOMAmer). SOMAmers are known in the art as single stranded DNA-based protein affinity reagents that are manufactured using a selection technology, for example, as described in: U. A. Ochsner et al., “Detection of Clostridium difficile toxins A, B and binary toxin with slow off-rate modified aptamers,” Diagnostic microbiology and infectious disease 76, 278-285 (2013); Ellington A D and Szostak J W, “In vitro selection of RNA molecules that bind specific ligands,” Nature 346, 818-22 (1990); Tuerk C and Gold L, “Systematic evolution of ligands by exponential enrichment: RNA ligands to bacteriophage T4 DNA polymerase,” Science 249, 505-10 (1990); Gold et al., “Aptamer-Based Multiplexed Proteomic Technology for Biomarker Discovery,” PLOS ONE 5(12): e15004 (2010); Davies D R, et al., “Unique motifs and hydrophobic interactions shape the binding of modified DNA ligands to protein targets,” Proc Natl Acad Sci USA 106:19971-76 (2012); Ramaraj T, et al., “Antigen-antibody interface properties: Composition, residue interactions, and features of 53 non-redundant structures,” Biochim Biophys Acta 1824, 530-32 (2012); Rohloff J C, et al., “Nucleic Acid Ligands With Protein-like Side Chains: Modified Aptamers and Their Use as Diagnostic and Therapeutic Agents,” Mol Ther Nuc Acids 3:e201 (2014), each of which is incorporated herein by reference. In an embodiment, the capture reagent is SOMA1. In an embodiment, the capture reagent is biotinylated SOMA1. SOMA1 can be immobilized on a solid support, for example, by (a) biotinylation of SOMA1 and binding of biotinylated SOMA1 to immobilized avidin, or (b) covalent coupling of amine-labeled SOMA1 to Costar 2525 amine-binding N-oxysuccinimide treated plates.

In one embodiment for measuring CXM, the detection reagent is mAb X34 and the capture reagent is SOMA1. In another embodiment for measuring CXM, the detection reagent is mAb X34 conjugated to at least one reporter molecule and the capture reagent is SOMA1. In another embodiment for measuring CXM, the detection reagent is mAb X34 conjugated to horseradish peroxidase and the capture reagent is SOMA1. In yet another embodiment for measuring CXM, detection reagent is a SOMAmer conjugated to horseradish peroxidase and the capture reagent is either the same or a different SOMAmer.

In an embodiment, the amount of collagen X marker in a sample may be quantified by (a) contacting the sample with immobilized SOMA1 so as to capture CXM bound to SOMA1 in a CXM-SOMA1 complex; (b) contacting the CXM-SOMA1 complex formed in step (a) with mAb X34 conjugated with a reporter molecule; and (c) detecting a reporter signal from the reporter molecule, wherein the reporter signal reflects the amount of CXM in the sample from the subject. In an embodiment, the amount of CXM in a sample, may be quantified by (a) contacting the sample obtained from the subject with biotinylated SOMA1 immobilized on a streptavidin-coated plate; (b) removing material in the sample not bound by SOMA1 in step (a); and (c) detecting immobilized CXM using mAb X34 conjugated with horseradish peroxidase (HRP), wherein an HRP signal reflects the amount of CXM in the sample obtained from the subject. In an embodiment, the amount of collagen X marker in a sample, may be quantified by (a) contacting the sample with immobilized SOMA1 so as to capture CXM bound to SOMA1 in a CXM-SOMA1 complex; (b) contacting the CXM-SOMA1 complex formed in step (a) with mAb X34 conjugated with a chemical flourophore; and (c) detecting a reporter signal from the chemical flourophore, wherein the reporter signal reflects the amount of CXM in the sample from the subject. In one embodiment, the chemical fluorophore is R-phycoerythrin. In yet other embodiments, the reporter molecule may be alkaline phosphatase, luciferase, a quantum dot fluorescent reporter molecule, a Raman reporter molecule, a Maverick Detection System reporter molecule, an electrochemical immunosensor reporter molecule, an aptosensor reporter molecule, a mass spectrometry reporter molecule, an sAB-colloidal gold conjugate reporter molecule, and/or a DNA-directed immobilization reporter molecule.

In an embodiment for measuring Cxm, the detection reagent is chicken anti-mouse-rNC1 and the capture reagent is SOMA1. In an embodiment for measuring Cxm, the detection reagent is a chicken anti-mouse-rNC1 antibody bound to an HRP-conjugated secondary antibody, and the capture reagent is SOMA1.

In an embodiment, the amount of Cxm in a sample may be quantified by (a) contacting the sample with immobilized SOMA1 so as to capture Cxm bound to SOMA1 in a Cxm-SOMA1 complex; (b) contacting the Cxm-SOMA1 complex formed in step (a) with an antibody conjugated with a reporter molecule; and (c) detecting a reporter signal from the reporter molecule, wherein the reporter signal reflects the amount of Cxm in the sample from the subject. In an embodiment, the amount of Cxm in a sample, may be quantified by (a) contacting the sample obtained from the subject with biotinylated SOMA1 immobilized on a streptavidin-coated plate; (b) removing material in the sample not bound by SOMA1 in step (a); and (c) detecting immobilized Cxm using an antibody conjugated with horseradish peroxidase (HRP), wherein an HRP signal reflects the amount of Cxm in the sample obtained from the subject. In an embodiment, the amount of collagen X marker in a sample, may be quantified by (a) contacting the sample with immobilized SOMA1 so as to capture Cxm bound to SOMA1 in a Cxm-SOMA1 complex; (b) contacting the Cxm-SOMA1 complex formed in step (a) with an antibody conjugated with a chemical flourophore; and (c) detecting a reporter signal from the chemical flourophore, wherein the reporter signal reflects the amount of Cxm in the sample from the subject. In one embodiment, the chemical fluorophore is R-phycoerythrin.

A standard curve can be generated by contacting a known quantity of a serially diluted rNC1 sample with biotinylated SOMA1 immobilized on a streptavidin coated plate, and then contacting the rNC1-SOMA1 complexes formed with a detection reagent. The detection reagent signal is proportional to the amount of rNC1 present in each serial dilution of the known rNC1 sample. The signals from the known serial dilutions are then used to generate a calibration curve using standard techniques, for example, a 4-parameter logistic regression curve. The signal generated by an unknown sample is then input into the calibration curve equation and the quantity of collagen X marker detected is the output.

Another aspect of the present invention is a method for measuring CXM to monitor the extent of bone growth response, wherein the measurements occur before, during, and/or after an intervention or treatment. Details regarding measuring the amount of CXM (or Cxm) are the same as those set forth with regard to the other embodiments described above. Measuring or determining the amount of CXM provides a real-time reading of bone growth plate activity that corresponds to skeletal bone growth velocity at the time when the sample was taken from the subject. In an embodiment, the amount of CXM is measured to monitor the bone growth response in a subject in need thereof to an intervention that is intended to stimulate bone growth, including but not limited to, growth hormone therapy, C-type natriuretic peptide (CNP) therapy, bone morphogenetic protein (BMP) therapy, insulin-like growth factor 1 (IGF-1) therapy, FGFR3 antagonist therapy, or vosoritide (BMN 111) therapy. In an embodiment, the amount of CXM is measured to identify the beginning and ending of the pubertal growth spurt as a means to guide the timing of idiopathic scoliosis intervention in a subject in need thereof, including but not limited to, bracing of the spine or surgical fusion of the spine. In an embodiment, the amount of CXM is measured to monitor the bone fracture healing of a subject having been diagnosed with a bone fracture. In an embodiment, the amount of CXM is measured to monitor or detect osteoarthritis in a subject in need thereof. In an embodiment, the amount of CXM is measured to monitor or detect cancer in a subject in need thereof. In an embodiment, the amount of CXM is measured to monitor or detect heterotopic ossification in a subject in need thereof. In an embodiment CXM is measured to monitor or detect other diseases or disorders, including but not limited to, rickets, hypogonadism, growth hormone deficiency, intrauterine growth retardation, Russell Silver Syndrome, or vitamin D deficiency. Any other intervention known in the art intended to stimulate bone growth can be similarly used in conjunction with the methods of the invention.

In an embodiment, the measurement of CXM or Cxm, as described herein, corresponds to bone growth plate activity which in turn is correlated with skeletal bone growth velocity at the time the sample was taken from the subject.

This invention provides a purified collagen X marker. This invention provides collagen X marker purified by a process comprising binding to an aptamer, such as SOMA1. This invention provides collagen X marker purified by a process comprising binding to an antibody, such as mAb X34. This invention provides collagen X marker purified by a process comprising binding to an aptamer and an antibody. This invention provides CXM purified by a process comprising binding to SOMA1 and mAb X34. The level of purity for a purified collagen X marker is determined relative to its purity in its natural state circulating in the bloodstream. Accordingly, this invention provides a purified collagen X marker that has been enriched, relative to its amount in a blood, serum, or plasma sample, by 10%, 30%, 100%, 300%, 1000%, 3000%, 10,000%, or more.

### Examples

Specific embodiments of the invention will now be demonstrated by reference to the following examples. It should be understood that these examples are disclosed solely by way of illustrating the invention and should not be taken in any way to limit the scope of the present invention.

All serum, plasma, and dried blood spot (DBS) samples were collected prospectively under protocols approved by the Institutional Review Board from children and adults between 2014 and 2017 from either Shriners Hospital for Children or from Oregon Health & Science University (OHSU) in Portland, Oreg. Patients from Shriners Hospital were enrolled for single appointments where serum, plasma, DBS and urine samples at the same time. Patients from OHSU were enrolled in a longitudinal study collecting serum and DBS at time points of approximately 0, 6, and 12 months. Sample sizes for tests of marker to growth velocity associations were determined by a priori power analyses using standard values for Type I error (α=0.05) and Type II error (β=0.2; hence power 1−β=0.8) to detect correlations of 0.4 or larger.

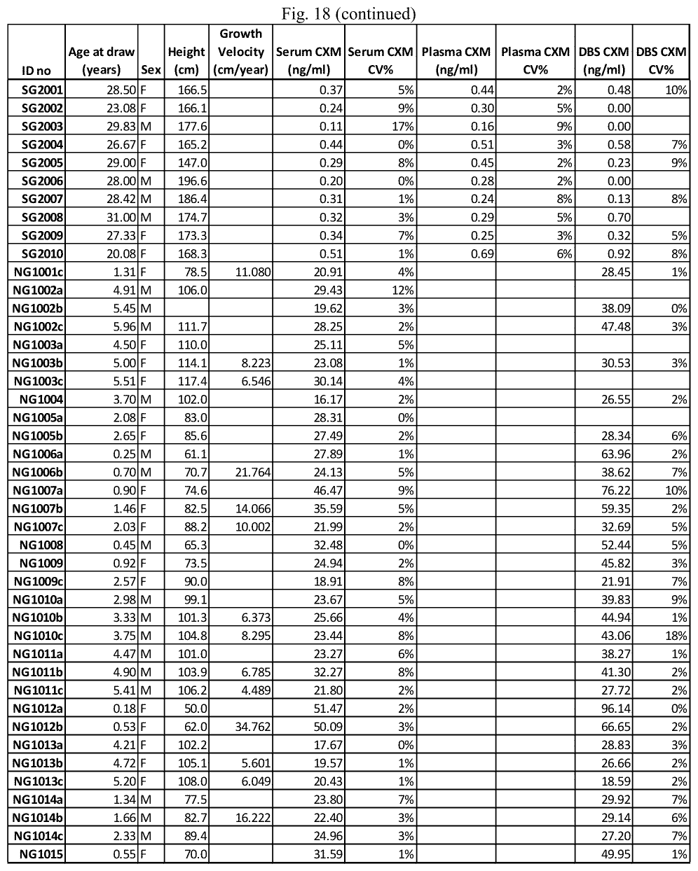

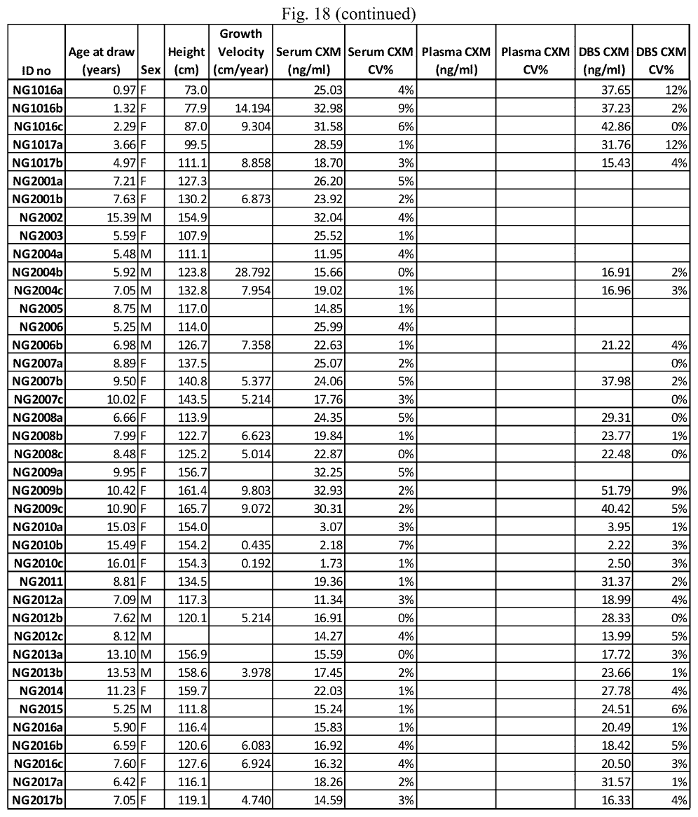

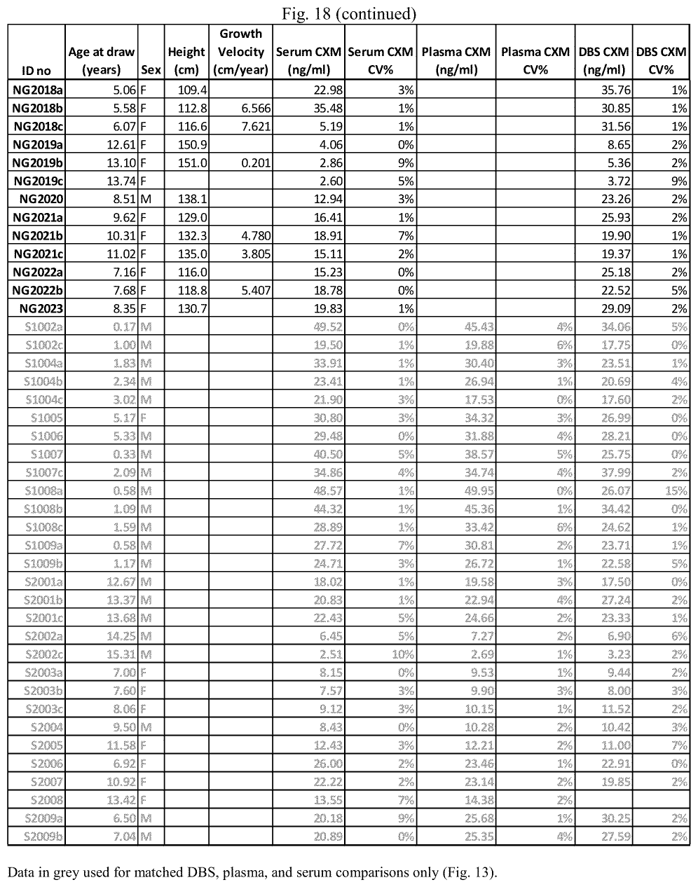

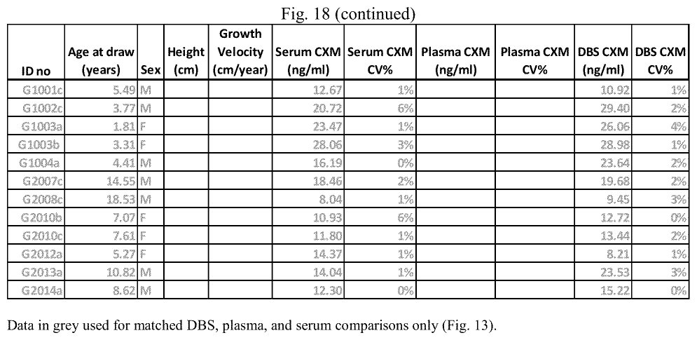

Heights were measured on an easy glide stadiometer (Perspective Enterprises) calibrated by a standard 100 cm rod. Measurements were done in a clinical setting in the Pediatric Endocrine and Diabetes clinics by a medical assistant specifically trained in accurate measurement techniques. Umbilical cord blood samples were obtained through the Oregon Cord Blood Donation Program at OHSU. Umbilical cord serum samples were purchased from BioReclamationIVT. Height, weight and arm span measurements were recorded at the time of sampling for each patient. Growth velocity was calculated using the change in height measurements from longitudinal samples collected. Plasma and serum samples were processed in vacutainers (Becton-Dickinson #368036 and #367983, respectively), aliquoted into microcentrifuge tubes, and stored immediately at −20° C. DBS samples were obtained by finger sticks and spotting onto Whatman 903™ Protein Saver Cards. DBS cards were then dried for 1-4 hours at room temperature, placed in re-sealable bags containing desiccant packets, and stored at −20° C. until assayed. All samples included in this study were assayed in a blinded fashion in duplicate. Information pertaining to these samples can be found in FIG. 18.

Samples for diurnal variation testing were obtained from well managed but otherwise healthy diabetic children ages 2-14 years enrolled in the OHSU Pediatric Diabetic Clinic. Patients prepared a DBS each time they stuck their finger for glucose measurements. The time and date were recorded and dried cards stored desiccated in a re-sealable bag in the dark at room temperature. Once sample collection was completed the cards were returned to Shriners Hospital for Children in envelopes satisfying mailing requirements provided by the Center for Disease Control. Upon arrival DBS cards were stored at −20° C. until assayed.

Samples for fracture healing were collected at the University of California, San Francisco (UCSF) Zuckerburg San Francisco General Hospital and Trauma Center. Fracture patients were enrolled within two weeks of experiencing a fracture. The fractures were documented radiographically and DBS samples were collected at initial appointment and at each checkup thereafter. DBS cards were then dried for 1-4 hours at room temperature, placed in re-sealable bags containing desiccant packets, and stored at −20° C. DBS cards were mailed in dry ice packages to Shriners Hospital in Portland, Oreg. for CXM concentration testing using standard DBS elution and testing protocols.

### Recombinant Proteins

Recombinant proteins to human and mouse NC1 regions were obtained from BioMatik (Human rNC1 #RPU140912, Mouse rNC1 #RPU140913).

Recombinant peptides had a polyhistidine tag
(SEQ ID NO: 1: MGHHHHHHSGSEF)
followed by the NC1 protein sequences:
Human: SEQ ID NO: 2:
TGMPVSAFTVILSKAYPAIGTPIPFDKILYNRQQHYDPRTGIFTCQIPGI
YYFSYHVHVKGTHVWVGLYKNGTPVMYTYDEYTKGYLDQASGSAIIDLTE
NDQVWLQLPNAESNGLYSSEYVHSSFSGFLVAPM
Mouse: SEQ ID NO: 3:
TGMPVSAFTVILSKAYPAVGAPIPFDEILYNRQQHYDPRSGIFTCKIPGI
YYFSYHVHVKGTHVWVGLYKNGTPTMYTYDEYSKGYLDQASGSAIMELTE
NDQVWLQLPNAESNGLYSSEYVHSSFSGFLVAPM

### Type X Collagen Antibodies

Human specific mouse monoclonal antibodies X34 and X53 were either conjugated to horseradish peroxidase (HRP, Southern Biotech) or covalently coupled to agarose using AminoLink Plus immobilization kit (Thermo #44894). Rabbit polyclonal antibodies (pAbs) were raised against both human and mouse rNC1 (USCNK #PAC156Hu01 or #PAC156Mo01) or a human NC2 peptide (LSBIO #LS LS-C157654). Ayes Labs Inc. prepared and purified a chicken polyclonal antibody to the mouse rNC1 sequence above (avian pAb raised to mouse rNC1). HRP-conjugated secondary antibodies included goat anti-rabbit (Amersham #NA934V) and goat anti-chicken (Ayes Labs Inc. #H-1004).

### Components for ELISAs

96 well EIA/RIA high-binding plate (Costar #3590). Immuno-pure streptavidin (Thermo #21125). Superblock blocking buffer (Thermo #37515). BSA for coating plates (RMBIO #BSA-BAF-01K). BSA for assay solutions (Gold Biotechnology #A-421-100). Tween-20 (Fisher #BP337-500). Dextran sulfate sodium salt (Sigma #31404-25G-F). Calibrators for assays were rNC1 proteins from BioMatik described above.

### ELISA Buffers

SBT buffer: 100 mM NaCl, 5 mM KCl, 10 mM hemisodium HEPES (pH 7.5), 0.05% Tween-20. SBTM: SBT buffer+5 mM MgCl₂. SBTE: SBT buffer+5 mM EDTA. Sample diluent: SBTM buffer+1% BSA and 1% Dextran Sulfate. Conjugate diluent: SBTM buffer+1% BSA. Coating buffer: 1.59 g Na₂CO₃/2.93 g NaHCO₃ in 1 L H₂0 (pH 9.6). PBST: Dulbecco's phosphate buffered saline+0.02% Tween. Blocking buffer: PBST+1% BSA. SOMAmer plating buffer: SBTE+1% BSA. Stop solution: 160 mM H₂SO₄.

### Other Buffers

TBST: Tris buffered saline+0.1% Tween. Gel loading buffer: sample buffer (Thermo #NP0007)+sample reducing agent (Thermo #NP0009). Low salt buffer: 1 mM HEPES (pH 7.5) 1 mM MgCl₂, 0.02% Tween. SOMAmer elution buffer: 20 m M ethanolamine (pH 10), 5 mM EDTA, 0.02% Tween.

### Other Components

Amicon Ultra Centrifugal filters (#UFC200324). Pierce streptavidin magnetic beads (Thermo #88816). Bolt antioxidant (Thermo #BT0005). Imperial protein stain (Thermo #24615). Pierce Top 12 Abundant protein depletion spin columns (Thermo #85165). AminoLink Plus immobilization kit (Thermo #44890). NuPage bis-tris and tris-glycine gels (Thermo). Human adiponectin (R&D Systems #1065-AP-050). Human C1q (abcam #ab96363). Human collagens type I and II (Abnova #P4915 and #P4916). Human collagen type VIII ca and a2 NC1 domains (Antibodies online #ABIN1079239 and #ABIN1098982).

### Identification of Marker in “Depleted” Cord Serum

After depletion of their most abundant serum proteins (using Thermo #85164 columns), umbilical cord and adult serum samples were concentrated on 3 kDa ultra-centrifugal filters and loaded on a 4-12% bis-tris gradient SDS-PAGE gel system (5 μl serum/lane). Full-length type X collagen from the medium of a HEK cell line developed by Wagner et al., “Coexpression of a and 13 subunits of prolyl 4-hydroxylase stabilizes the triple helix of recombinant human type X collagen,” Biochem J 352 (pt. 3) 907-911 (2000), was used as a positive control. The separated proteins were transferred to nitrocellulose at 56 volts for 1 hour, blocked in TBST+3% BSA for 1 hour, and probed with HRP-X34 (anti-NC1) and HRP-X53 (anti-C1) at a 1:5,000 dilution, or a polyclonal anti-NC2 at 1:1,000 dilution followed by an HRP-conjugated secondary. Antibody incubations were in TBS-T+1% BSA for 1 hour.

### Immunoprecipitation, Aptoprecipitation and Western Blot Procedures

All precipitations were performed overnight at 4° C. with end to end turning. Immunoprecipitation with mAb X34-agarose (10 μl of 50% slurry for each 5 ml of serum) was performed in PBST, after which the agarose beads were washed 5× in the PBST. Trimeric marker was eluted from mAb-X34 by moderate heating in gel loading buffer (70° C. for 10 minutes). Monomeric subunits were generated by eluting beads with 100 mM acetic acid (˜pH 2.5) followed by lyophilization of the eluate and resuspension of protein in gel loading buffer. Aptoprecipitations were performed with SOMA1-magnetic beads (2.6 nmoles of biotinylated SOMA1/10 mg of streptavidin magnetic beads) using 5 μl of a 10 mg/ml bead solution per 5 μl of serum diluted into SBTM. Beads with bound marker were washed 3× with SBTM and eluted in a small volume of SOMAmer Elution Buffer (pH 10) before adding to gel loading buffer. Following SDS-PAGE, proteins were transferred to nitrocellulose at 56 volts for 1 hour at 4° C. The blots were then blocked with 3% BSA in TBST, washed and probed as described.

### Purification of Marker

Cord plasma was obtained after centrifugation of donated cord blood samples, and each unit received 4.18 ml of 1M MgCl₂, 2 ml of 1 M hemisodium HEPES, and 2 ml of 100 mM sodium EGTA. Then 6 ml of 10% dextran sulfate was added slowly with stirring to prevent formation of a Mg⁺⁺/dextran sulfate precipitate. This preparation was placed on ice, stirred slowly for 1 hour and spun at 8,000 g for 1 hour. The resulting supernatant was distributed into 50 ml tubes, with 1.7 mg of SOMA1-magnetic beads (see above) per tube. The tubes were turned end over end overnight, after which the magnetic beads were collected into 1.5 ml conical tubes and washed sequentially with: SBTM (3×1 ml)), SBTM+4 M NaCl (4×1 ml), SBTM (1×1 ml), and low salt buffer (2×1 ml). Elution of CXM was performed by adding 100 μl of SOMAmer elution buffer to the pooled beads and shaking on an orbital mixer for 10 minutes at RT. The resulting supernatant was highly enriched in CXM in its native trimeric form. At this point four volumes of SBTM with elevated HEPES (50 mM/pH 7.5) was added to neutralize the sample for long-term storage.

### Mass Spectrometry

CXM was purified from 6 units of cord plasma (˜250 ml) according to the procedure described above. To concentrate, denature and dissociate CXM subunits, 400 μl of the marker in SOMAmer Elution Buffer was directly precipitated with 10% TCA, acetone washed, and dried for 10 minutes at 96° C. The dried pellet was dissolved in 20 μl of Gel Loading Buffer and heated at 96° C. for 10 minutes. Two lanes of a 12% NuPage Bis-Tris gel were loaded for SDS-PAGE (Bolt Antioxidant was added to the upper tank buffer to reduce in-gel oxidation). One lane, containing 5% of the sample, was subsequently blotted and probed with the anti-NC1 USNCK pAb to determine the position of CXM on the gel. The other lane, containing the remaining 95%, was directly stained with colloidal Coomassie Blue, and the corresponding region excised. This gel fragment was digested with Protease Max+Trypsin and analyzed on a Thermo Scientific Orbitrap Fusion Mass Spectrometer. Collagen X peptides were identified using the Sequest data analysis program, for example, as described in Eng, et al., “A face in the crowd: Recognizing peptides through database search,” Mol Cell Proteomics 10, R111.009522 (2011), and T. Alan, A. C. Tufan, “C-type natriuretic peptide regulation of limb mesenchymal chondrogenesis is accompanied by altered N-cadherin and collagen type X-related functions,” J Cell Biochem 105, 227-235 (2008), both of which are incorporated by reference herein. Data analysis was performed within the Proteome Discoverer software suite (Thermo Scientific). Sequest HT was used to search MSMS spectra against a June 2016 version of the human Swiss-Prot database, and Percolator filtered resulting peptide matches to an overall false discovery rate of 1%. The 307 high confidence identifications of type X collagen presented had an average cross correlation (XCorr) of 3.5 and an average delta mass of 0.79.

### Development of SOMAmer Capture Reagent for CXM

The recombinant human NC1 region described above was biotinylated and submitted to Somalogic Inc. for “SELEX” affinity capture of potential high affinity SOMAmers (slow off-rate modified aptamers). FIG. 2B indicates that the recombinant peptide was in its native trimeric form. Before performing SELEX selection, the following proteins were pre-adsorbed to the SOMAmer library to avoid potential cross reactivity: human collagen types I, II, and VIII, and the serum proteins adiponectin and complement C1q. Ten high affinity SOMAmers were generated, of which the highest affinity form (SOMA1; 160 pM) gave the best response when used in a sandwich assay with HRP-conjugated mAb X34. Several of these SOMAmers and their characteristics are listed below:

| | Affinity | Ability to | Compatibility |
|---|---|---|---|
| | to rNC1 | bind CXM | with mAb X34 |
| B-15653-9_3 (SOMA1) | 1.6E−10 | +++++ | +++++ |
| B-15653-127_3 | 3.9E−10 | +++++ | + |
| B-15653-65_3 | 8.7E−10 | +++++ | +++ |
| B-15648-115_3 | 1.0E−09 | +++++ | +++ |
| B-15653-30_3 | 1.1E−09 | +++ | ++ |
| B-15661-18_3 | 2.2E−09 | ++ | ++ |

As noted, B-15653-9_3 (i.e., SOMA1) was chosen due to its high affinity for CXM (160 picomolar) and its low steric hindrance of mAb X34 binding. As further shown above, B-15653-127_3, B-15653-65_3, B-15648-115_3, B-15653-30_3, and B-15661-18_3 also showed high affinity for CXM but somewhat less compatibility with mAb X34.

### Assay Procedure

1) Sample incubations. Calibrators, controls and samples were prepared in Sample diluent and aliquoted into “SOMA1 capture” assay plates. All sample, detector and reporter incubations were at 100 μl/well and performed at 37° C. with shaking at 450 rpm. The SOMA1 reagent described proved effective at capturing both human and mouse markers, so the procedure below was used to generate plates for both assays. STREPTAVIDIN: 100 μl/well of streptavidin (4 μg/ml in Coating Buffer) was added to each well of a 96 well ‘High Bind’ ELISA plate and incubated overnight at 4° C. Wash the next day with PBS (3×300 μl/well). BLOCK 1: Plates were blocked for 1 hour by adding 300 μl/well of Blocking Buffer at RT and washed with PBS (3×300 μl/well). SOMA1: 100 μl/well of biotinylated SOMA1 (3 pmoles/ml in SOMAmer Plating Buffer) was added to plates and incubated overnight at 4° C. Wash the next day with PBS (5×300 μl/well). BLOCK 2: Plates were blocked for 10 minutes with 300 μl/well of Superblock at RT, emptied, and patted dry on paper towels to remove excess Superblock. DRY: The plates were then dried in a desiccator at RT (until desiccator environment reached less than 10% humidity). STORAGE: Plates were individually sealed in foil bags with desiccant pouches and stored at 4° C. until use.

2A) Human assay detector incubation. Plates were washed 3× with SBTM, patted dry, and incubated with HRP-conjugated mAb X34 (1:5,000 in conjugate diluent) for 1 hour.

2B) Mouse assay detector/reporter incubations. Plates were washed 3× with SBTM and incubated with chicken anti-mouse-rNC1 (5 μg/ml in Conjugate Diluent) for 1 hour. Plates were washed 5× with SBTM and incubated with HRP-conjugated secondary (1:5,000 dilution in Conjugate Diluent) for 1 hour.

3) Develop and Read. Plates were washed 3× with SBTM, tapped dry, and developed with TMB substrate at room temperature. After 10 minutes the reaction was stopped by adding 50 μl of stop solution and brief mixing on a shaker at 650 rpm. The OD 450 was read within 30 minutes of stop solution addition.

### ELISA Assay Calibrators and Controls

The rNC1 from BioMatik was reconstituted per instructions. Absolute concentration was initially determined using a Qbit 2.0 Fluorimeter from Invitrogen and confirmed by amino acid analysis using a Hitachi L-8800A. Calibrators were prepared by diluting rNC1 to 800 pg/ml in Sample Diluent and serial dilution to 12.5 pg/ml. QC controls were created by diluting rNC1 into sample diluent to concentrations of 700, 250, and 10 pg/ml, respectively. Serum and plasma samples from normally growing children were diluted 1:200 in Sample Diluent. Quality control of inter-assay and intra-assay determinations was monitored using matrix-specific (serum, plasma, or DBS) rNC1 spiked controls at low, medium, and high concentration levels along with full calibration curves for each ELISA plate. Assays were deemed valid if QC replicates were <20% intra-assay CV % and within +/−20% of inter-assay assigned concentration (except for rNC1 QC 10 pg/mL (LOW) due to its low concentration).

### DBS Elution Procedure

One 3.1 mm punch was taken per pediatric DBS spot and eluted with 250 μl of Sample Diluent in the well of a sealed polypropylene microplate. Due to low CXM concentration, adult samples utilized 2 punches. The plate was incubated overnight at 4° C. on ice to reduce condensation. Finally, the elution plate was then placed on a shaker at 450 rpm for 10 minutes at room temperature. Each sample (100 μl) was assayed in duplicate and concentration determined from a serial diluted rNC1 calibrator curve using 4 Parameter Logistic (4PL) nonlinear regression model fit from BioTek Gen5 software (R²>0.95 was acceptable). DBS quality controls of 70, 30, and 1 ng/ml were also added to wells of the elution plate for assay validity. Each result was multiplied by their associated dilution (calculated dilution factor assumes 1.67 μl plasma per spot assayed) for their equivalent ng/ml concentration. This dilution factor may need to be adjusted in the future based on assay concentration comparisons of DBS versus serum values for matched samples, for example, as referred to in T. W. McDade et al., “High-sensitivity enzyme immunoassay for C-reactive protein in dried blood spots,” Clin Chem 50, 652-654 (2004), which is incorporated by reference herein.

### Comparison of Growth Velocity to Cxm Levels in Mouse

DBS samples were obtained from mice 2, 3, 4, 6, 8, 10 and 12 weeks old. Following blood collection mice were euthanized and the lengths of tails and dissected femurs and tibias were measured with calipers. Femur and tibia measurements were averaged from both limbs. Individual growth rates were derived by the following formula. Change in length=(length measurement of individual)−(average length of all individuals at previous time point). Growth Velocity=Change in length/elapsed time between measurements. Elution and measurement of DBS Cxm was performed according to procedures described above.

### Half-Life Testing

Two male and three female 25 week old FVB-8 mice, with 0-1.5 ng/ml baseline levels of endogenous Cxm were injected intravenously with 532 ng of mouse rNC1 into their tail veins. Blood was sampled from tail or saphenous veins at roughly 10, 30, 60, 120, and 240 minutes after injection. The Cxm concentration determined for the 10 minute time point was set at 100%. Subsequent sampling and concentrations were plotted as a percentage of the initial value for each mouse in the study.

### CXM Stability Testing

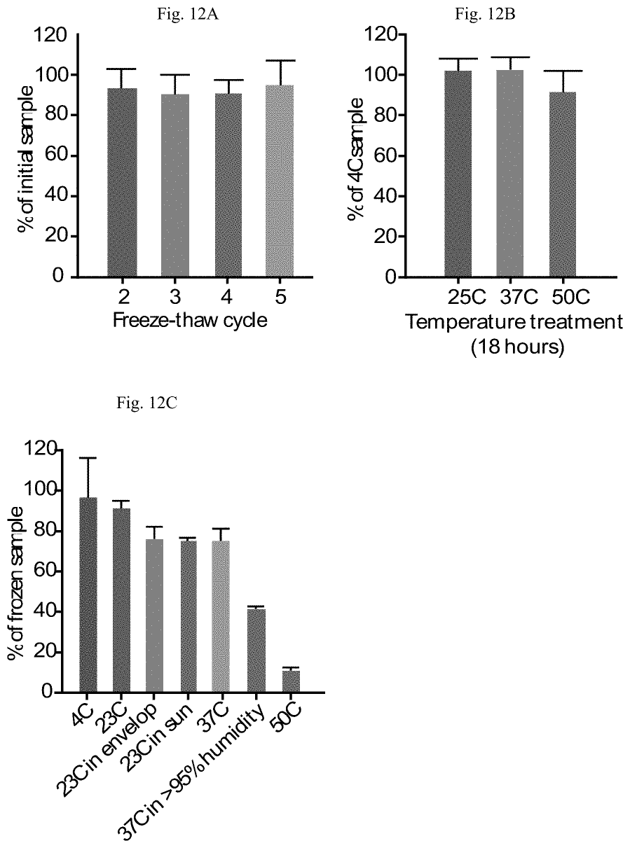

For freeze/thaw analysis five separate serum samples from children in our study 1.6-12 years of age were thawed and assayed by CXM ELISA for an initial determination. Samples were then re-frozen at −20° C. for 18 hours, thawed, and sampled again. This process was repeated for 5 freeze-thaw cycles, as shown in FIG. 12A. CXM concentrations for each subsequent freeze-thaw step were compared to the initial value and percent recovered plotted. Percentage of recovery for serum samples cycled through 5 freeze/thaws with first freeze/thaw sample used as standard for comparison (n=5).

For temperature stability analysis cord serum, serum, and plasma samples were thawed, aliquoted, and incubated at 4° C., 25° C., 37° C., or 50° C. conditions for 18 hours. As shown in FIG. 12B, samples were then assayed by CXM ELISA and the result for each temperature treatment was compared to their respective 4° C. measurement.

DBS stability analysis utilized Whatman 903™ protein saver cards spotted with umbilical cord blood. Dried cards were placed in re-sealable bags with desiccant and stored for 8 days at −20° C., 4° C., 23° C. (on bench), 23° C. in envelope (on bench), 23° C. (in variable sunlight, on windowsill), at 37° C., at 37° C. in a cell culture incubator (card placed in a petri dish instead of the re-sealable bag, no desiccant, in >95% humidity controlled, 5% CO₂ incubator), and at 55° C. As shown in FIG. 12C, after incubation 3.1 mm punches were eluted and assayed by CXM ELISA and the resulting concentrations were compared as a percentage of the −20° C. measurement.

### Statistical Analysis

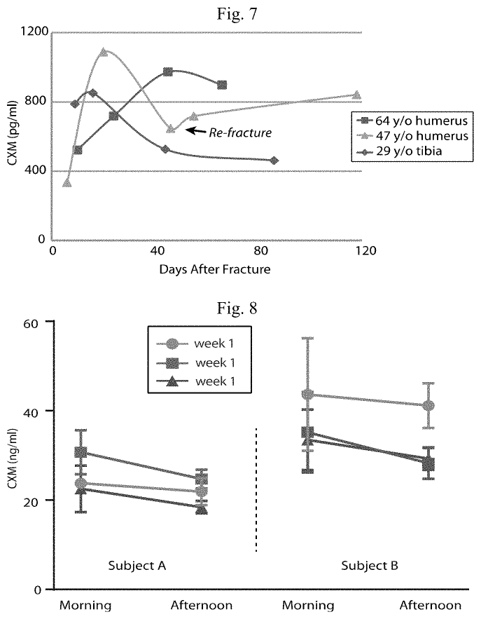

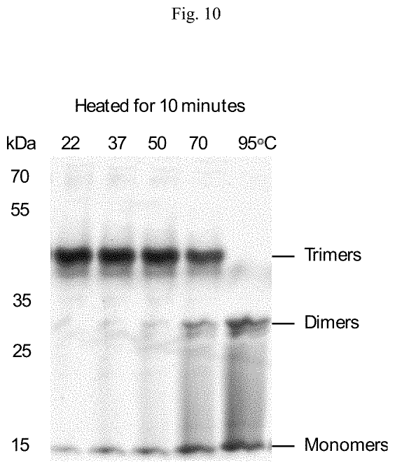

Across the mouse and human samples, CXM was plotted against age to show growth curves and with superimposed established growth velocity curves for comparison for humans. For tests of association of CXM with growth velocity, scatterplots and linear fit summary lines were generated, and Pearson's correlation and statistical significance was calculated. A power series fitted summary line was generated to summarize the non-linear relationship of CXM to growth velocity in healthy children. FIG. 7 is a plot showing CXM concentration measured at different time points after acute long bone fractures in a 29 year old male (diamond) and in 47 (triangle) and 64 year old (square) females. Arrow indicates re-fracture in the 47 year old patient. The criterion p-value was set at p<0.01 for all tests of significance. This study tested a small number of theoretically targeted relationships, so no adjustment was made of criterion p-values for multiple comparisons. All statistical analysis was performed using GraphPad Prism 7 and Stata 14. Lower limit of quantitation calculations were performed using statistical equations published by D. A. Armbruster et al., “Limit of blank, limit of detection and limit of quantitation,” Clin Biochem Rev 29, S49-52 (2008), which is incorporated by reference herein. Our lower limit of blank (LOB) for the CXM assay was determined to be 0.0722 ABS units at 450 nm. Lower limit of quantitation (LLOQ) testing (FIG. 10) was performed by diluting human rNC1 calibrator to concentrations of 7.5, 6.25, 4.5, and 3.13 pg/ml and running 16 separate replicates in a CXM ELISA. FIG. 10 depicts 3 ug/lane of the recombinant NC1 region of mouse type X collagen was analyzed on SDS-PAGE after incubation in gel loading buffer for 10 minutes at the indicated temperatures. Protein was visualized with Coomassie stain. From this data we were able to calculate the theoretical limit of detection (LOD) as 0.0837 ABS units at 450 nm and the LLOQ as 0.1139 ABS units at 450 nm. This LLOQ value equates to 5.4 pg/ml.

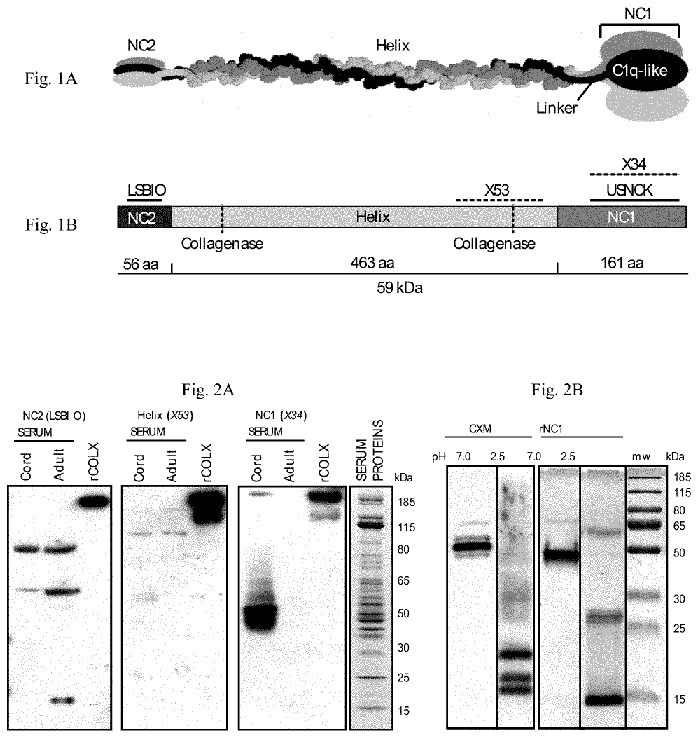

Type X collagen is a homotrimeric protein with non-collagenous amine and carboxy termini (NC2 and NC1 regions, respectively) connected by a triple helical collagenous domain (FIG. 1). As shown in FIG. 1A, Non-collagenous N-terminal (NC2) and C-terminal (NCI) domains are connected by a collagenous triple helix. The NC1 domain is subdivided into a compact “C1q-like” region that resolves in the crystal structure, and a “linker” region that does not. FIG. 1B depicts the schematic of antibody binding regions and collagenase sites. Solid lines indicate peptide sequences to which polyclonal antibodies (pAbs) were raised. Hatched lines indicate regions within which X53 and X34 monoclonal antibodies bind. Also shown are two sites susceptible to collagenase cleavage. To identify which of these domains may be present in blood we compared umbilical cord serum, where type X collagen concentration should be high, to adult serum, where expression should be much lower. SDS-PAGE/western blot analysis of cord versus adult sera was performed after specific immunodepletion of the most abundant serum proteins. FIG. 2A depicts western blots of umbilical cord serum, adult serum and full length rCOLX (positive control). Equivalent blots of 4-12% gels were probed with antibodies to the non-collagenous NC2 domain (left panel), collagen helix (center panel) and non-collagenous NC1 domain (right panel). The fourth panel of FIG. 2A depicts representative Coomassie stain of serum proteins present in cord and adult lanes. FIG. 2B depicts in the left panel a western blot of immunoprecipitated CXM eluted at pH 7.0 versus pH 2.5, separated on a 12% gel, and probed with a pAb (USCNK) to the NC1 domain. In the right panel of FIG. 2B depicts rNC1 separated by SDS-PAGE before (left lane) or after (right lane) pH 2.5 treatment and stained for protein. FIG. 2A shows that recombinant full-length type X collagen (rCOLX) was detected by the probes for each region, but only the NC1-specific probe monoclonal antibody (mAb) X34 could readily detect proteins in cord serum that were visually absent in adult serum. Because mAb X34 only detects multimeric forms of the NC1 domain, the ˜50 kDa NC1 region detected in FIG. 2A, right panel, most likely consists of carboxy-terminal trimers. Directly probing blots of serum was considered preferable for this initial screen. However, the high concentration of protein in the serum samples (see last panel of 2A) caused the NC1-specific signal to be less well-defined compared to affinity purified samples (FIG. 2B) and produced several non-specific cross-reactions with the NC2 and helix antibodies.

When the putative marker was immunopurified with immobilized mAb X34, eluted with moderate heat, and probed with a polyclonal antibody (pAb) that recognizes both monomeric and multimeric NC1 regions, the same principal 50 kDa band was observed (FIG. 2B, left panel, 1st lane). However, when the immunoprecipitated marker was eluted with acetic acid (˜pH 2.5) and probed with the same pAb, lower molecular weight bands of ˜17, 19 and 23 kDa were detected (FIG. 2B, left panel, right lane), consistent with their being component subunits of a denaturation-resistant trimeric protein. For comparison, SDS-PAGE of recombinant trimeric NC1 (rNC1) before and after acetic acid treatment (FIG. 2B, right panel), yielded similar peptides of ˜50 kDa and 15 kDa, respectively.

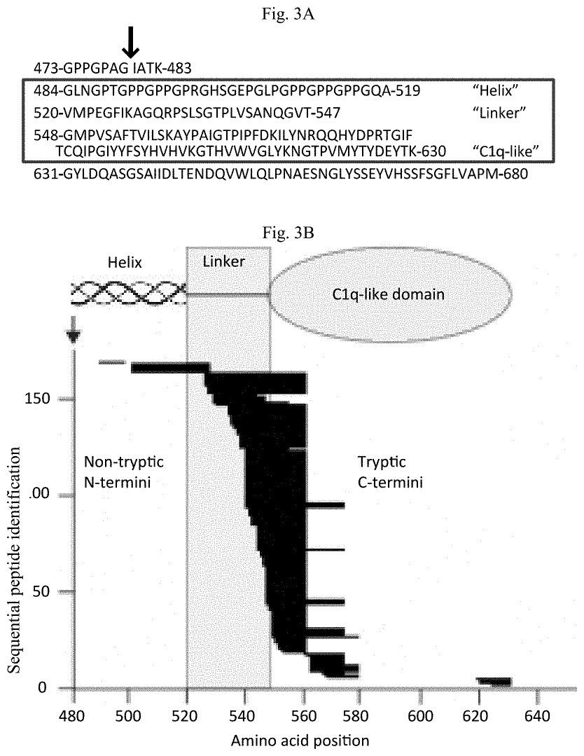

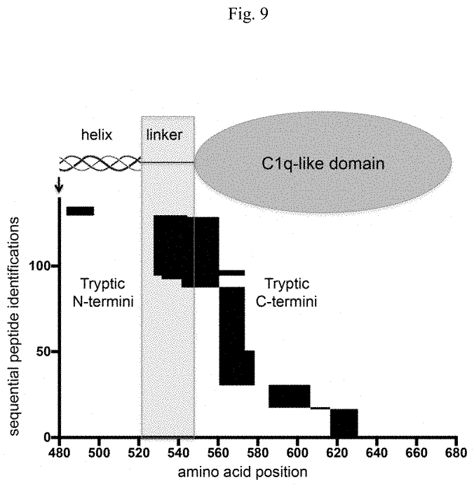

Mass spectrometry of purified/trypsinized marker confirmed its identity. The boxed portion of FIG. 3A indicates the region defined by high-confidence peptides identified in mass spectrometry analysis. The amino acids which are depicted above the box of FIG. 3A are amino acids immediately upstream of identified region that include the proposed collagenase cut site. The lack of a tryptic cleavage site within the C-terminal last 50 amino acids of type X collagen (G631-M680) made this peptide too large to be detected. FIG. 3B depicts semi-tryptic high-confidence peptide sequences identified by mass spectrometry, represented by stacked horizontal lines corresponding to their placement within the CXM marker. Proposed collagenase cut site corresponds to amino acid position 480. Functional domains are diagrammed above graph with the linker region defined by a shaded box. FIG. 9 provides a graph of peptides whose N and C termini are both tryptic. Tryptic high-confidence peptide sequences are represented by stacked horizontal lines corresponding to their placement within the CXM marker. Proposed collagenase cut site, as shown in FIG. 9, corresponds to beginning of X-axis. Functional domains are diagrammed above graph with the linker region defined by a shaded box. All high confidence sequences mapped from the end of the C1-helix through most of the NC1 domain (G484 to K630—FIG. 3A). A total of 129 peptides identified resulted from tryptic cleavage at both N and C termini (FIG. 9). A total of 168 semi-tryptic peptides had non-tryptic N-termini, presumably present in the purified marker before trypsinization (FIG. 3B) while only 10 had non-tryptic C-termini. Most of the non-tryptic N-termini localized to the 28 amino acid “linker” region between the C1 triple helix and the “C1q-like domain.” This suggests that the marker is initially released by collagenase activity at a previously proposed site (G479, as discussed in T. M. Schmid et al., “Type X collagen contains two cleavage sites for a vertebrate collagenase,” Journal of Biological Chemistry 261, 4184-4189 (1986), which is incorporated by reference herein) in the C-terminal part of the triple helical domain, just upstream of the sequence identified here. Additional cleavages then occur in the “linker” region while the compactly coiled C1q-like trimer resists further proteolysis. The size range of such fragments, containing the entire C1q domain and variable portions of the attached linker and collagenous regions is consistent with the subunit sizes previously identified by western blotting (FIG. 2B, left panel, right lane). Trimers composed of these variably lengthened fragments would then account for the multiple bands shown in FIG. 2B, left panel, left lane. We designated this group of human NC1 trimeric domains with frayed ends as CXM.

### CXM Abundance Varies by Age and Sample Source

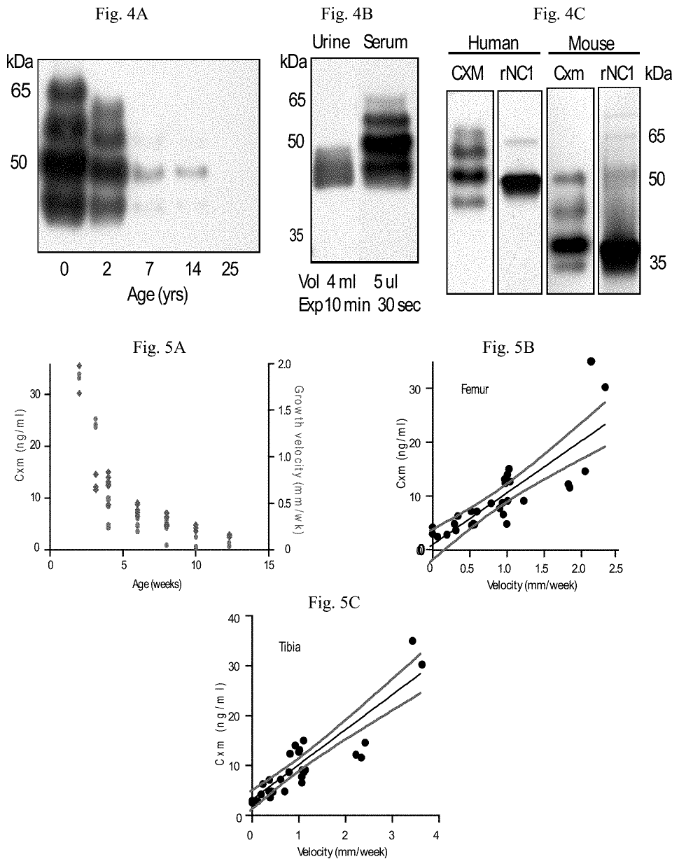

If the occurrence of CXM in blood was an indicator of cartilage turnover in growth plates, its concentration in blood would be expected to decrease with age as growth velocity slows. Equivalent serum volumes obtained from cord blood (t=0), and subjects 2, 7, 14 and 25 years of age were “aptoprecipitated” using a SOMAmer (slow off-rate modified aptamer). Aptoprecipitation has been described in, for example, in U. A. Ochsner et al., “Systematic selection of modified aptamer pairs for diagnostic sandwich assays,” Biotechniques 56, 125-128, 130, 132-123 (2014); J. C. Rohlof et al., “Nucleic Acid Ligands With Protein-like Side Chains: Modified Aptamers and Their Use as Diagnostic and Therapeutic Agents,” Mol Ther Nucleic Acids 3, e201 (2014), each of which are incorporated by reference herein. Aptoprecipitation is analogous to immunoprecipitation, except that an aptamer reagent (SOMAmer) is used instead of an antibody. This SOMAmer, hereafter referred to as SOMA1, was selected against human rNC1 but recognizes both native human and mouse isoforms. SDS-PAGE/western blot analyses of the aptoprecipitates were then probed with human-specific mAb X34. FIG. 4 depicts western blots of CXM aptoprecipitated with SOMA1 and probed with mAb X34 including FIG. 4A which is the western blot of serum of individuals of increasing ages (0 yrs.=umbilical cord serum). FIG. 4B is matched urine and serum samples from a 2 month old infant (Vol=volume of sample, Exp=exposure time for autoradiography). FIG. 4C is aptoprecipitated trimeric markers from human serum (CXM) or mouse serum (Cxm) probed with pAbs raised against their respective recombinant NC1 domains, and compared to Coomassie-stained gels of the same recombinant proteins (rNC1). Here, the CXM signal dropped progressively with the age of the subject and became undetectable in the 25 year old adult sample (FIG. 4A); however the pattern of bands remained the same irrespective of the subject's age.

An analysis comparing serum and urine obtained from a single 2 month old infant (FIG. 4B) showed that only low molecular weight marker components were detected in urine. However, its concentration in urine was 26,000 fold lower than in serum. In FIG. 4C the mouse trimeric serum marker (Cxm) showed a pattern of bands similar to the human CXM, but migrated approximately 10 kDa further down the gel. Correspondingly, recombinant mouse NC1, which is trimeric (see FIG. 10), showed the same 10 kDa shift. The reason for this mobility difference is not clear, however, the presence of an extra negative charge in the mouse NC1 sequence may contribute.

### Marker Analysis in Mice Age 1-12 Weeks

The feasibility of using the new marker as an indicator of bone growth velocity was tested in wild type mice by plotting serum Cxm concentration against age and the growth velocities of the tail, femur and tibia. Cxm concentration was measured in a sandwich ELISA that used SOMA1 and avian pAb for capture and detection, respectively. FIG. 5A depicts Cxm serum concentration and the growth velocity of mouse tails were plotted against age of mice (n=29). FIGS. 5B and 5C depict Cxm serum concentrations were plotted against matched femur (B) or tibia (C) growth velocities (n=29), with linear fit lines and 95% CI (confidence interval). Respective Pearson's correlations are: femur r=0.82, p<0.0001; tibia r=0.89, p<0.0001. FIG. 5A shows that C×m values dropped substantially through the first few weeks in a pattern similar to the decrease in calculated velocity of tail growth. In addition, correlations were obtained when the growth velocities calculated from femur and tibia measurements of individual mice were plotted against their Cxm concentrations (FIGS. 5B and C).

### Marker Analysis in Healthy Infants and Children

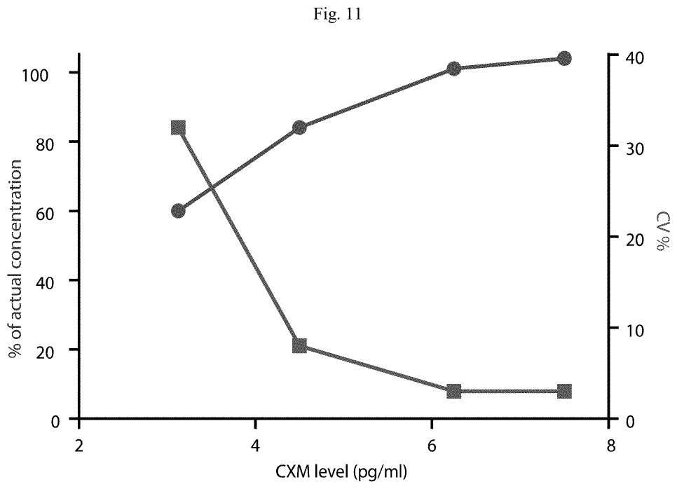

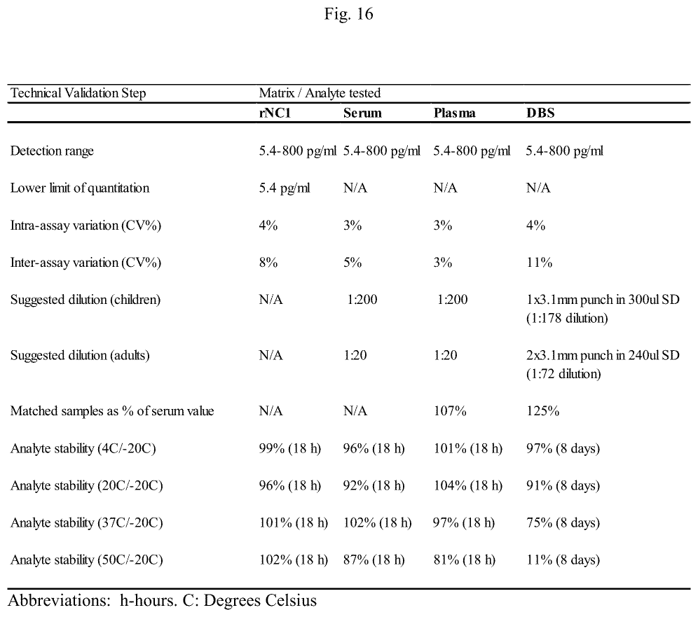

A human CXM ELISA assay similar to the mouse Cxm assay was developed using SOMA1 for capture and mAb X34 for detection. FIG. 16 summarizes the performance characteristics of this marker assay. FIG. 11 plots the lower limit of quantitation for CXM. LLOQ testing was performed by diluting rNC1 calibrator to extremely low levels and calculating concentration CV % for each level (square plot). Concentrations determined for each sample were plotted as a percentage of their actual concentration (circle plot). Notably, it is sensitive to 5.4 pg/ml (FIG. 11), allowing for accurate CXM determinations with extremely small volumes of blood, and the CXM marker exhibits stability over a variety of storage conditions (FIG. 12). Overall intra-assay coefficient of variation (CV %) of blood samples is on average below 5%, with similarly low inter-assay variations.

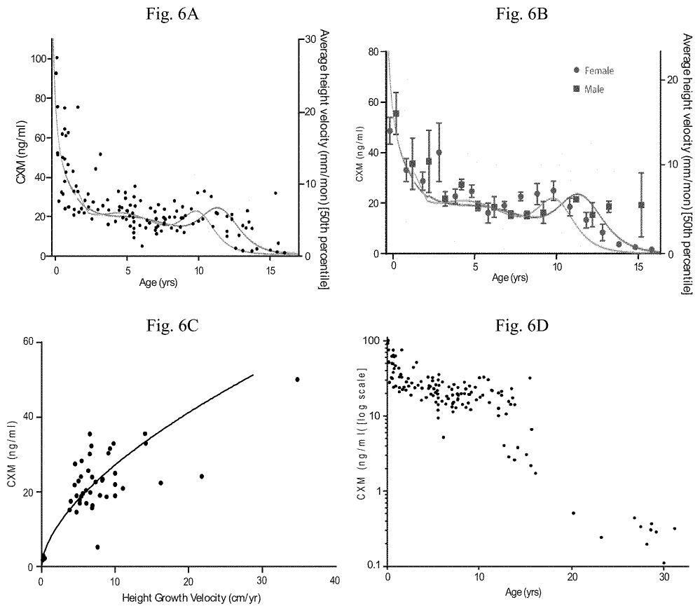

In accordance with local Institutional Review Board approval and after the nature and possible consequences of the studies were explained, serum samples obtained from 83 normally growing, healthy infants and children ranging in age from birth to 18 years were assayed for CXM and compared. As shown in FIG. 6A, Serum CXM is plotted against age for normally growing infants and children (n=129). Established height velocity curve averages for males and females are superimposed for comparison. FIG. 6B is CXM is plotted against age, grouped by sex and shown as mean+/−standard error (SE). Sex matched velocity norms for males and females are superimposed as before. FIG. 6C depicts infants and children 0.18-16 years of age were measured for length/height and assayed for serum CXM at 0, 6, and 12 month periods (n=44). Height velocities were calculated as change in length/height over time interval, converted to cm/year and plotted against CXM (adjusted R2[weighted]=0.88, p<0.001). FIG. 6D is the Log—transformed CXM serum concentrations for normally growing children and non-growing adults are plotted against age (N=139).

To maximize sample size, we relaxed the assumption of independence and included observations for normally developing children who were measured 2 or 3 times (mean=2.125) at 6 month intervals (n=40) along with 43 normally developing children and 10 adults who were measured once. Established growth velocity curves for infants and children of both sexes are superimposed on FIGS. 6A and 6B for reference. Male and female CXM concentrations were not statistically different when prepubertal age groups were compared (FIG. 6B) (Centers for Disease Control and Prevention, 2000, National Center for Health Statistics, CDC growth charts: United States (http://www.cdc.gov/growthcharts)). However, the concentrations varied more during pubertal years and differed between males and females, presumably reflecting the variability in timing of pubertal growth spurts. These cross-sectional data document that CXM concentrations parallel well established growth velocity standards commonly used to evaluate childhood growth.

### Human Growth Velocity Measurements

Longitudinal height data and blood samples collected at approximately 6 month intervals from 26 individuals allowed CXM concentration to be plotted against annualized height velocity (FIG. 6C). To maximize sample size, we relaxed the assumption of independence and included two growth velocity observations for 14 children along with 12 with only one observation. A non-linear power series algorithm was used to fit data with the respective coefficient of determination shown. The linear correlation of CXM and height velocity was more modest in this sample (Pearsons r=0.66, p<0.001, 95% confidence interval: 0.45 to 0.80) than in the mouse samples, but fitting a non-linear power series line improved the correlation of our marker to height velocity in humans (adjusted R² [weighted]=0.88). The observed association is consistent with our model that the concentration of the marker reflects growth plate activity and the rate of skeletal growth, however the sample size was too small to confidently fit a curved function to the data.

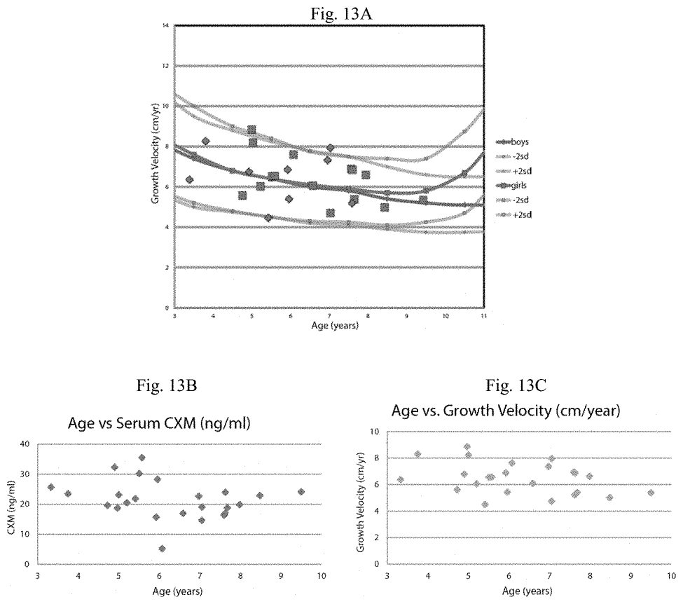

To document that our study population was growing normally, we plotted stadiometer-based height velocities of 23 subjects between the ages of 3.3 and 9.5 years against established norms for this age group (FIG. 13A), also noted in J. M. Tanner et al., “Clinical longitudinal standards for height and height velocity for North American children,” J Pediatr 107, 317-329 (1985), which is incorporated by reference herein. This age range was used because growth is typically relatively steady. With exception of two subjects who plotted slightly beyond 2 standard deviations (SD), our subjects fell within 2 SD of the norms indicating that our population was not skewed.

It is difficult to directly compare CXM-based estimates of height velocity to stadiometer-based (observed) height velocity determinations because they measure different parameters of growth. To gain insight into this issue, we plotted C×M values and observed height velocities against age and visually compared their relative dispersion (FIGS. 13B and 13C). Both FIGS. 13B and 13C show a slight decline with age. This comparison showed less dispersion for the observed velocity measurements than CXM, suggesting observed measurements may be better for accurately determining height velocity averaged over several months; however, it is unlikely that CXM would be used for this purpose.

### CXM in Healthy Adults

In contrast to growing children, CXM concentrations dropped to around 300 pg/ml on average in adults. To show the full range of C×M values, CXM concentrations from 10 healthy, non-growing 20-30 year old adults were plotted on a logarithmic scale with the younger subjects previously mentioned (FIG. 6D). CXM appears to level off in healthy adults at concentrations well below those of growing children.

### CXM in Adult Fracture Healing

Bone fractures heal through endochondral ossification during which type X collagen-containing fracture callus is degraded and replaced by bone, similar to what occurs in the growth plate. The rate of healing and amount of callus vary by fracture severity, how well the healing fracture is stabilized, and the size of bone that is fractured. Most likely the relative amount of CXM released from a single or even a few fractures would be less than the amount released from all growth plates in a growing skeleton, so our assay would be unlikely to detect minute changes in CXM concentrations in children with fractures. In adults, low endogenous concentrations of CXM may allow for monitoring fracture healing using the CXM marker. Preliminary evidence shows that a temporal pattern in which CXM rises, peaks and then falls during fracture healing can be detected in adults (FIG. 7). This temporal pattern is consistent with the “endochondral” phase of fracture healing, which typically occurs from 1-3 weeks after initial fracture. The 47 year old female subject in this figure offers a unique window into the proposed relationship between CXM and fracture healing. This individual's initial fracture was associated with a peak in CXM at 20 days post-fracture, but she then experienced a proximal re-fracture, which was associated with another rise in CXM that corresponded temporally to radiographic evidence of secondary fracture callus. Comparison of the temporal patterns of C×M during fracture healing of the 64 year old versus the 29 year old subjects is consistent with the notion that healing may occur more slowly with aging. See also, C. Lu et al., “Effect of age on vascularization during fracture repair,” J Orthop Res 26, 1384-1389 (2008), and D. P. Taormina et al., “Older age does not affect healing time and functional outcomes after fracture nonunion surgery,” Geriatr Orthop Surg Rehabil 5, 116-121 (2014), each of which are incorporated by reference herein.

### Serum Versus Plasma Versus DBS

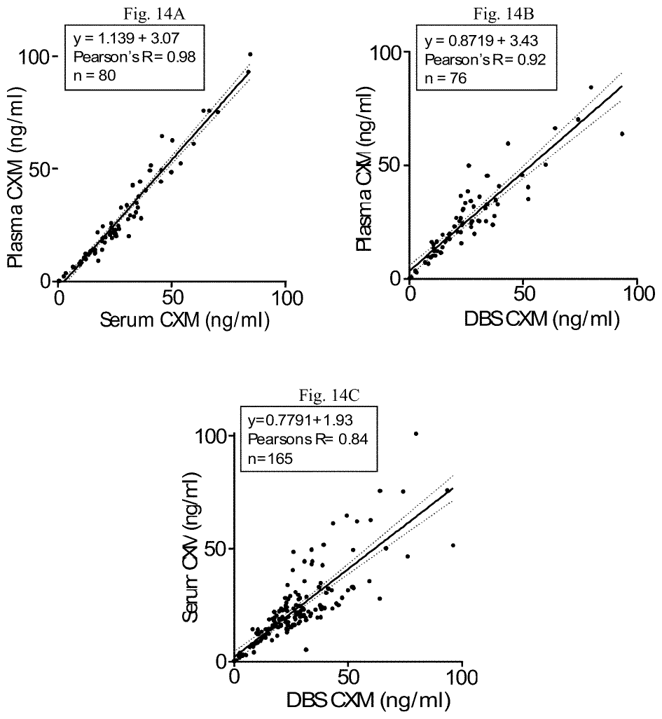

Our marker ELISA was developed using serum, but in many instances, only plasma or DBS samples are available, which have been shown to give equivalent results in other marker assays, as discussed in T. W. McDade et al., “High-sensitivity enzyme immunoassay for C-reactive protein in dried blood spots,” Clin Chem 50, 652-654 (2004), which is incorporated by reference herein. To determine the suitability of these alternative blood samples for CXM we compared concentrations of the marker in subjects whose blood was collected as serum and plasma; or serum, plasma, and DBS simultaneously. Eighty paired serum and plasma samples were collected and assayed, and CXM results for plasma showed slightly higher values on average (+7%) compared to their paired serum counterparts (FIG. 14).

When comparing paired serum versus DBS or plasma versus DBS samples, the matched concentrations suggest that DBS may be more comparable to plasma rather than serum. The Pearsons r for plasma versus DBS was better than that for serum versus DBS at 0.92 versus 0.84, respectively. DBS average readings tended to be higher on average with higher variability versus both serum and plasma. Given the potential variations inherent in DBS sampling procedure and extraction compared to venipuncture it is not surprising we observed more variability with our DBS samples. Despite these variability issues, analysis of the extracted DBS gave comparable results to our matched serum and plasma samples, with FIG. 14 plotting the best-fit linear regression line and CI.

### Biologic Variation

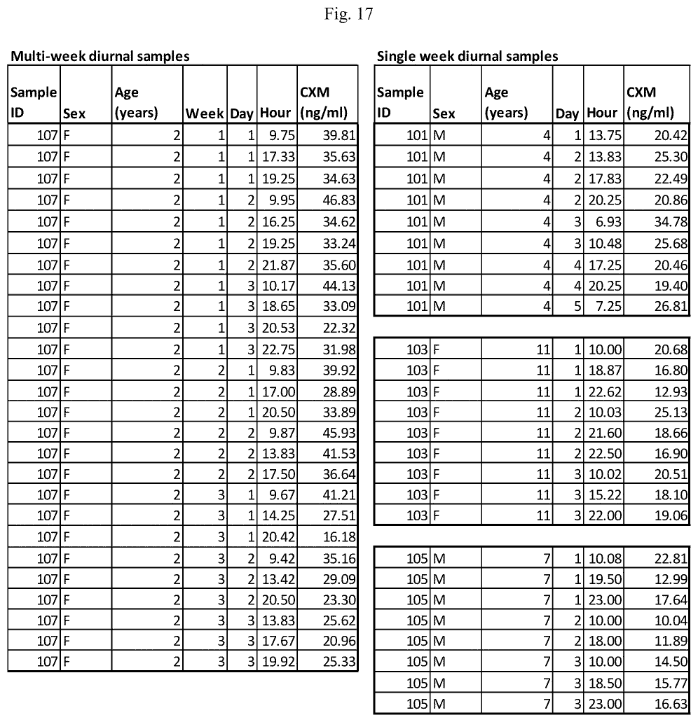

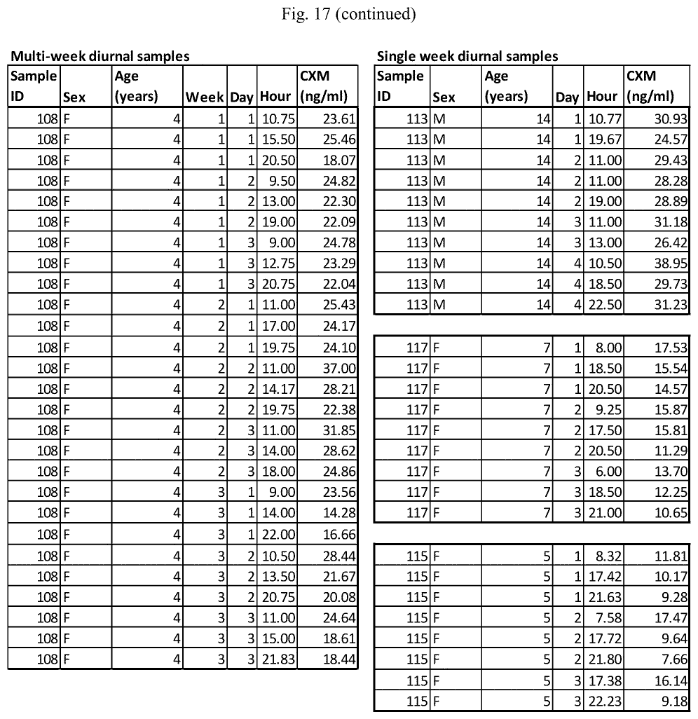

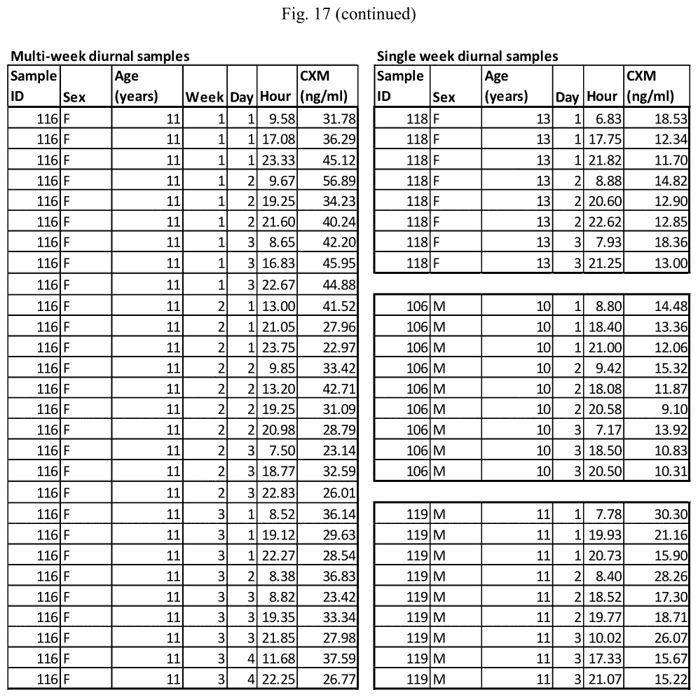

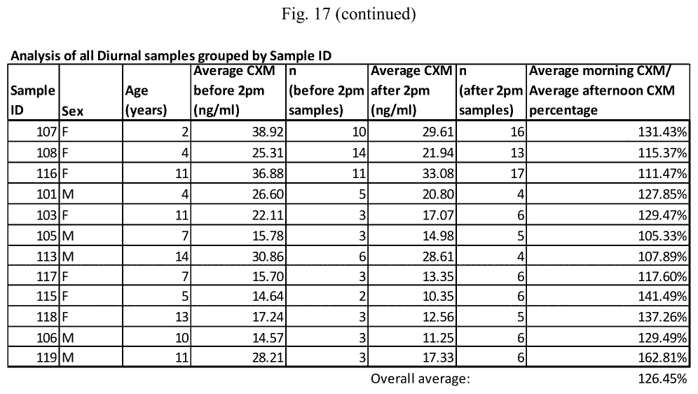

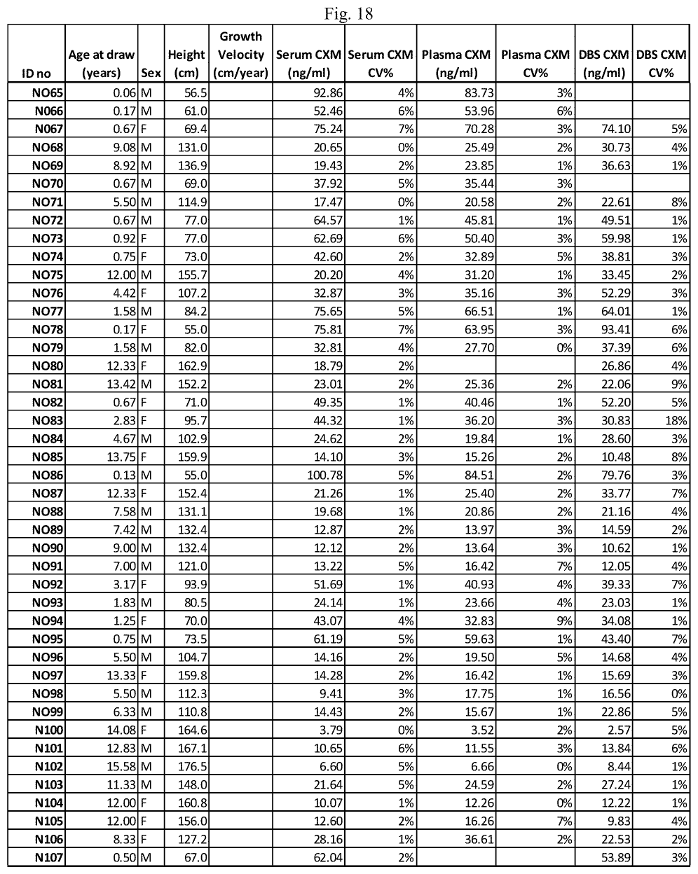

Many markers exhibit diurnal variation. To determine if CXM shows such variation, we measured CXM in 12 normally growing children ages 2-14 years with well-controlled diabetes. DBS cards were spotted and the time recorded coincident with finger stick for glucose monitoring. Sampling was at least three times a day for three consecutive days and in some cases for three consecutive weeks. Using 2 pm as cutoff for morning and afternoon samples, CXM concentrations were on average 26% higher before 2 pm than after 2 pm (data shown in FIG. 17). FIG. 8 illustrates this pattern and modest weekly variation is shown from two girls sampled over 3 weeks. Subject A was a 4 year old female and Subject B was an 11 year old female each tested morning and afternoon for three consecutive weeks (n=27 and n=28, respectively). Average CXM concentration readings and SD were plotted.

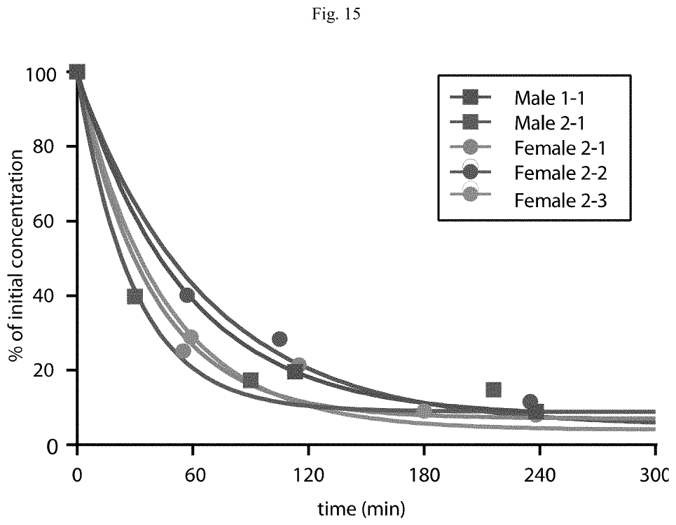

To assess the stability of CXM/Cxm in the circulation, mouse rNC1 was injected intravenously into 25 week old mice and blood samples were assayed at various times up to 240 minutes following injection (FIG. 15). Half-life of rNC1 was determined by injecting mouse rNC1 into 5 adult mice. Time zero corresponds to the initial blood sample 10 minute after injection. Best fit curves for each mouse were created in Prism using non-linear fit of one-phase decay. The results suggest CXM/Cxm has a half-life of approximately 30 minutes.

The results indicate that CXM, the intact trimeric NC1 domain of type X collagen, escapes degradation in the skeletal growth plate and can be detected in blood, where its concentration reflects overall growth plate activity in the body and correlates with velocity of skeletal growth. As such, this degradation by-product of skeletal growth behaves as a real-time marker for linear skeletal growth velocity and has many potential clinical applications.

### CXM Identification, Characterization and Assay

The synthesis of type X collagen is normally restricted to the hypertrophic zone of the skeletal growth plate, where it is secreted into cartilage matrix during the latter stages of endochondral ossification in all growing bones. This matrix serves as a template for bone growth during which degradation proceeds until growth stops following adolescence. The interface between the hypertrophic zone and newly formed bone—ossification front—is highly enriched in extracellular proteolytic enzymes engaged in degrading and removing hypertrophic cartilage matrix as the ossification front expands and the bone lengthens. The enzymes known to possess collagenase activity, which are thereby candidates for type X collagen degradation, include matrix metalloproteinase 13 (MMP13) secreted from terminally differentiated hypertrophic chondrocytes, MMP9 from osteochondroclasts and proteases released from vascular cell precursors invading the cartilage template from the bone marrow, for example, see, N. Ortega et al., “Matrix remodeling during endochondral ossification,” Trends Cell Biol 14, 86-93 (2004), which is incorporated by reference herein.

Type X collagen has two proposed collagenase cleavage sites in its helical domain (FIG. 1). The ˜50 kDa size of the predominant fragment detected by western blot suggests that CXM is the product of the carboxy collagenase cleavage plus additional cleavage events that trim the fragment to smaller sizes. The mouse Cxm appears to undergo cleavages similar to the human CXM. Detection of distinct bands slightly larger and smaller than the predominant 50 kDa human CXM band combined with the mass spec results implies there are favored cleavage sites at the amino terminal end of the C-terminal collagenase cleavage fragment. Our attempts to identify the cleavage sites by N-terminal sequencing have been unsuccessful to date.

The mouse studies, as described herein, indicate that the CXM marker in vivo half-life is relatively short, ˜30 minutes. In contrast, the marker is very stable in vitro, in isolated serum, plasma, and DBS samples. For example, CXM displays <10% degradation in serum for 18 h at 37° C., (FIG. 12), can undergo multiple freeze thaws, and resists degradation at temperatures above freezing. The ability of the marker to resist proteolysis likely reflects its compact molecular configuration (see, O. Bogin et al., “Insight into Schmid Metaphyseal Chondrodysplasia from the Crystal Structure of the Collagen X NC1 Domain Trimer,” Structure 10, 165-173, which is incorporated by reference herein). CXM's resistance to serum proteases and low urinary excretion suggests another clearance pathway is involved. Trimeric adiponectin, a circulating hormone that is both genetically closely related to type X collagen and structurally similar to CXM, is rapidly cleared by the liver with a very similar half-life, for example, as discussed in N. Halberg et al., “Systemic fate of the adipocyte-derived factor adiponectin,” Diabetes 58, 1961-1970 (2009), which is incorporated by reference herein, indicating that CXM may be removed through a similar mechanism.

Analysis of paired serum, plasma, and DBS samples showed that CXM concentrations were similar across sample types, although plasma and DBS readings tended to be slightly higher on average than serum values (FIG. 16). Differences in marker concentrations have been shown in matched biological sample types, for example, see, M. Dupin, T et al., “Impact of Serum and Plasma Matrices on the Titration of Human Inflammatory Biomarkers Using Analytically Validated SRM Assays,” J Proteome Res 15, 2366-2378 (2016), incorporated by reference herein. The DBS determinations were on average closer to those of the plasma samples rather than serum, suggesting that plasma may be the preferred choice of blood specimens for this assay. Of note, the overall inter- and intra-assay variation of plasma and serum samples was comparable.

### Clinical Relevance

It is well established that growth velocity is highest in young infants, drops substantially over the first two to three years, remains relatively low during childhood, increases modestly during the pubertal growth spurt and drops to zero after the spurt is complete. The scatter plot of our cross-sectional serum data from healthy infants and children shows a similar trend (FIG. 6A). Our numbers represent the first attempt to relate CXM to established human growth data, and they provide a strong indication that the marker levels reflect skeletal growth velocity.

CXM represents a real-time read-out of growth plate activity that corresponds to instantaneous skeletal growth velocity at the time of sampling in contrast to average growth velocity calculated from measuring incremental growth over several months, typically 6 months or more. As such, no comparable marker exists for CXM validation. If growth were a slow, steady and constant process, one would expect the real-time and average velocities to be very similar. However, if growth varies from day to day or even by time of day, as our data suggest, the two might not agree. Similarly, CXM may not necessarily predict length or height, both of which reflect accumulated growth in contrast to CXM, which measures growth rate at a single point of time. Despite these caveats, both mouse Cxm and human C×M values correlate with velocities calculated from measured interim growth, suggesting that variability must not be too great.

The correlation of CXM to growth velocity in human subjects was higher using a non-linear power curve (adjusted R² [weighted]=0.88, p<0.001) rather than a linear best fit (Pearson's r=0.66, p<0.001) that was used with the mouse data. FIG. 6 included some participants with more than one data observation. The relaxation of the assumption of independence might lead to narrower sample variability and risk modest inflation of the association of CXM and growth velocity. With a larger data set it may be found that a linear fit is more appropriate for plotting growth velocity versus CXM concentration, however the strong correlation from our data set demonstrates that CXM has the potential to provide estimates of growth velocity with narrow margins of error. CXM appears to be an informative, real-time indicator of skeletal growth velocity that has considerable potential benefit for the clinical management of skeletal growth and its disorders.

CXM-based estimates of height velocity may be compared to conventional stadiometer-based height velocity determinations. Each technique measures different parameters of growth, instantaneous growth velocity versus growth velocity averaged over 6-12 months, respectively. Consequently, they have different clinical applications and different utilities. For example, stadiometer-based methods will be most useful for cross-sectional, long-term studies. In contrast, CXM measurements may be most useful for assessing responses of individual children to interventions that affect growth in days to a few weeks. The difference is analogous clinically to the difference between measuring serum glucose and hemoglobin A1c in diabetic patients. The former measures glucose concentration at the time of sampling; the latter is an indicator of glucose metabolism over ˜3 months (R. R. Little et al., “The long and winding road to optimal HbA1c measurement,” Clin Chim Acta 418, 63-71 (2013), incorporated by reference herein). Both are used in the management of diabetes but for different purposes; the utility of one marker does not diminish the utility of the other.

CXM marker may be used for monitoring the growth response of poorly growing infants and children to interventions designed to improve growth. Examples include growth hormone and C-type natriuretic peptide derivative therapies for infants and children with growth hormone deficiency and achondroplasia, respectively. Compared to cross-sectional studies, the infant or child serves as his/her own control in this setting minimizing person-to-person variation. It is likely that treatments that directly or indirectly improve growth begin to act on the bone growth machinery within days or a few weeks at the least and that resulting changes in growth velocity could be detected by measuring CXM within this time frame assuming baseline concentrations were determined. Information about how an infant/child responds to treatment a month after initiation would be a substantial advantage over the current practice of waiting 6 months or more for growth velocity information. Being able to detect responses to therapeutic interventions in a much shorter time frame would greatly facilitate adjusting and comparing therapeutic interventions in these instances. It would also provide a new tool to investigate in depth how the skeleton responds to growth promoting interventions. Similarly, CXM testing may facilitate assessing and comparing the efficacy of programmatic interventions developed to alleviate malnutrition and other chronic diseases that negatively impact growth in resource-restricted regions of the world.

As described herein, testing of healthy diabetic children indicates that CXM exhibits diurnal variation with values highest in the morning, which would be consistent with the notion that diurnal factors, such as growth hormone, drive bone growth (K. L. Gamble et al., “Circadian clock control of endocrine factors,” Nat Rev Endocrinol 10, 466-475 (2014), which is incorporated by reference herein). Alternatively, diurnal variation of CXM could simply reflect loading (rising from bedtime horizontal position to daytime upright stature forces CXM from the growth plate into subchondral blood vessels) (see also, M. Lampl et al., “Saltation and stasis: a model of human growth,” Science 258, 801-803 (1992); C. Heinrichs et al., “Patterns of human growth,” Science 268, 442-447 (1995), incorporated by reference herein).

CXM may serve as a valuable tool to investigate short term variations in bone growth and their relationship to conventional parameters of growth.

Many of the growth plates that contribute to blood C×M values may not contribute to skeletal length or height, so one might argue that linking it to linear growth may not represent a perfect correlation. However, we believe the largest and most active growth plates in the body, namely those in the proximal and distal femurs and tibias, as well as the less active growth plates of the vertebral bodies, are likely to contribute most of the measurable CXM. Moreover, the correlations we detect for CXM versus length/height velocity and remarkable similarities of plotting CXM versus age to curves that plot clinically determined growth velocity to age argue that CXM is a useful indicator of linear bone growth.

The CXM marker has potential applications beyond those directly related to bone growth. For example, the management of idiopathic scoliosis frequently involves bracing and surgical fusion of the spine (T. Kotwicki et al., “Optimal management of idiopathic scoliosis in adolescence,” Adolesc Health Med Ther 4, 59-73 (2013), incorporated by reference herein). In both cases, the timing of intervention depends on the timing of the pubertal growth spurt; bracing takes advantage of the spurt, whereas surgical fusion is done after the spurt is finished. Frequent CXM testing could be used to guide the timing of both interventions.

Long bone fractures heal through endochondral ossification during which type X collagen-containing fracture callus is degraded and replaced by bone much like that which occurs in the growth plate, although the rate is influenced by other factors such as fracture severity, site, and stabilization (T. A. Einhom et al., “Fracture healing: mechanisms and interventions,” Nat Rev Rheumatol 11, 45-54 (2015), which is incorporated by reference herein). The data shown in FIG. 7 are preliminary but they show that CXM concentrations increase temporarily during the time frame when fractures would be expected to heal. They also lend evidence to the fact that our assay is sensitive enough to detect small changes over baseline CXM levels in adult subjects. Furthermore, these data support the concept that CXM is an indicator of endochondral ossification.

Articular chondrocytes often terminally differentiate (hypertrophy) in osteoarthritis (OA) raising the possibility that type X collagen could be used as a marker of OA activity (see also, M. B. Goldring et al., “Emerging targets in osteoarthritis therapy,” Curr Opin Pharmacol 22, 51-63 (2015), which is incorporated by reference herein). Indeed, low levels of type X collagen have been detected in sera from adults with severe OA (Y. He et al., “Type X collagen levels are elevated in serum from human osteoarthritis patients and associated with biomarkers of cartilage degradation and inflammation,” BMC Musculoskelet Disord 15, 309 (2014), incorporated herein by reference). The reported concentrations (24-128 pg/ml) are about 3 orders of magnitude lower than those we detect in growing infants, yet within the detectable limits of our assay. The epitope for the assay developed by these investigators maps to the NC1 domain of type X collagen. It is possible the mAb reported in this publication detects the same NC1 fragment reported here, although no biochemical studies were done to characterize the antibody target.

Type X collagen has been linked to cancer in two publications. In X. Sole et al., “Discovery and validation of new potential biomarkers for early detection of colon cancer,” PLoS One 9, e106748 (2014), which is incorporated by reference herein, it was detected by ELISA in sera of adult patients with colon cancer. The authors speculated that Runx2, a known transcriptional regulator of COL10A1 expression, is responsible for type X collagen production in the tumors. The second report, K. B. Chapman et al., “COL10A1 expression is elevated in diverse solid tumor types and is associated with tumor vasculature,” Future Oncol 8, 1031-1040 (2012), which is incorporated by reference herein, detected expression of COL10A1 mRNA by microarray analysis in diverse cancer types but not in normal tissues. Immunostaining of breast cancer tissues localized it to blood vessels suggesting that its expression is associated with vascular invasion of tumors. These reports raise the possibility that CXM could also be used as a marker for cancer detection in adults.

Throughout the specification various publications are referred to or cited, each of which is incorporated by reference herein in its entirety for all purposes.

1. A method for determining bone growth velocity comprising: (a) measuring an amount of CXM in a sample obtained from a subject in need thereof; and (b) comparing the amount of CXM measured in step (a) with a CXM standard curve, wherein the amount of CXM is measured using a combination of SOMA1 and mAb X34 as CXM-binding reagents.

2. The method of claim 1, wherein the sample is a blood sample, a serum sample, a plasma sample, or a dried blood spot.

3. The method of claim 1, wherein the subject is a human.

4. The method of claim 1, wherein SOMA1 and mAb X34 are used to bind CXM in a solid phase binding assay.

5. The method of claim 4, wherein the solid phase binding assay uses SOMA1 as a capture reagent and mAb X34 as a detection reagent.

6. The method of claim 5, wherein the capture reagent is immobilized on a solid phase support.

7. The method of claim 5, wherein the detection reagent is linked to a reporter molecule, further wherein the reporter molecule is selected from the group consisting of horseradish peroxidase (HRP), alkaline phosphatase, luciferase, a chemical fluorophore, a quantum dot fluorescent reporter molecule, a Raman reporter molecule, a Maverick Detection System reporter molecule, an electrochemical immunosensor reporter molecule, an aptosensor reporter molecule, a mass spectrometry reporter molecule, an sAB-colloidal gold conjugate reporter molecule, and a DNA-directed immobilization reporter molecule.

8. The method of claim 1, wherein the amount of CXM measured provides a real-time readout of bone growth plate activity that is correlated with skeletal bone growth velocity at the time of sampling.

9. A method for monitoring the extent of a bone growth response to an intervention intended to stimulate bone growth comprising measuring CXM in a pediatric human subject in need thereof before and after the intervention.

10. The method of claim 9, wherein CXM is further measured during the intervention.

11. The method of claim 9, wherein the intervention intended to stimulate bone growth is growth hormone therapy, C-type natriuretic peptide (CNP) therapy, bone morphogenetic protein (BMP) therapy, insulin-like growth factor 1 (IGF-1) therapy, FGFR3 antagonist therapy, or vosoritide (BMN 111) therapy.

12.-19. (canceled)

20. A method for detecting CXM in a sample obtained from a subject comprising capturing CXM using SOMA1 and detecting CXM using mAb X34.

21. The method of claim 20, wherein SOMA1 is immobilized on a solid phase support.

22. The method of claim 20, wherein mAb X34 is conjugated with a reporter molecule.

23.-36. (canceled)

37. The method of claim 20, wherein CXM or Cxm is detected in a multiplex format.

38.-40. (canceled)

41. The method of claim 7, wherein the chemical fluorophore is R-phycoerythrin.

42. The method of claim 1, wherein the amount of CXM measured in the sample from the subject is used to determine whether an intervention to treat a disease, disorder or condition is having a desired therapeutic effect.

43. The method of claim 42, wherein the intervention is selected from the group consisting of growth hormone therapy, C-type natriuretic peptide (CNP) therapy, bone morphogenetic protein (BMP) therapy, insulin-like growth factor 1 (IGF-1) therapy, FGFR3 antagonist therapy, and vosoritide (BMN 111) therapy.

44. The method of claim 42, wherein the disease, disorder or condition is selected from the group consisting of rickets, hypogonadism, growth hormone deficiency, intrauterine growth retardation, Russell Silver Syndrome, vitamin D deficiency, idiopathic skeletal hyperostosis, osteoporosis, and cancer.

45.-53. (canceled)
